(19) United States
(12) Patent Application Publication
(10) Pub. No.: US 2022/0033910 A1
(43) Pub. Date: Feb. 3, 2022

(54) Method for Detecting Liver Diseases

(71) Applicants: The General Hospital Corporation, Boston, MA (US); President and Fellows of Harvard College, Cambridge, MA (US)

(72) Inventors: David T. Ting, Dover, MA (US); Irun Bhan, Brookline, MA (US); Kelly Mosesso, Boston, MA (US); Martin Aryee, Cambridge, MA (US)

(21) Appl. No.: 17/274,955

(22) PCT Filed: Sep. 11, 2019
(86) PCT No.: PCT/US19/50532

§ 371 Date: Mar. 10, 2021

Related U.S. Application Data
(60) Provisional application No. 62/729,787, filed on Sep. 11, 2018.

(51) Int. Cl. C12Q 1/6886; G01N 1/34; G01N 33/58; G01N 33/50; G01N 33/68

(52) U.S. Cl. CPC C12Q 1/6886; G01N 1/34; G01N 33/582; G01N 33/5005; G01N 2800/56; C12Q 2600/112; C12Q 2600/158; G01N 2800/085; G01N 2800/52; G01N 33/6893

(57) ABSTRACT

The present invention relates to methods for diagnosing, determining, or monitoring liver diseases and conditions based on the blood concentration of circulating epithelial cells in and their gene expression.

### FEDERALLY SPONSORED RESEARCH OR DEVELOPMENT

This invention was made with Government support under Grant Nos. DK007191, EB012493, CA172738, and DK078772 awarded by the National Institutes of Health.

The Government has certain rights in the invention.

### TECHNICAL FIELD

This invention relates to a method of detecting and characterizing liver diseases in a subject by isolating and analyzing circulating epithelial cells (CECs).

### BACKGROUND

Liquid biopsy refers to sampling cellular material that originated from a solid organ and has entered the bloodstream. Circulating epithelial cells (CECs) can be detected by liquid biopsy in the setting of localized cancer (Stott S L, et al. Sci Transl Med 2010; 2:25ra23; Lucci A, et al. Lancet Oncol 2012; 13:688-95) and even preneoplastic pancreatic lesions (Rhim A D, et al. Gastroenterology 2014; 146:647-51; Franses J W, et al. Oncologist 2017) suggesting their presence is not exclusive to carcinogenesis.

Isolating CECs is a technological challenge due to their rarity in the bloodstream and the variable expression of antigens used for cell capture. For example, the EpCAM-dependent Veridex platform yielded Hepatocellular carcinoma (HCC) CEC detection rates of only 35% and 410% in two independent studies (Kelley R K, et al. BMC Cancer 2015; 15:206; Sun Y F, et al. Hepatology 2013; 57:1458-68). To overcome this limitation, an antigen-agnostic cell sorting device called the iChip, has been developed which isolates CECs while preserving cell viability and high-quality RNA content. The iChip device has previously been combined with an RNA signature based on established liver-specific markers to create an assay for the enrichment and detection of CECs in HCC (Kalinich M, et al. Proc Natl Acad Sci USA 2017; 114:1123-1128).

Other approaches to non-invasive diagnosis of HCC has been unsuccessful in achieving high detection rate. For example, a recent study has shown that detection of HCC by combining cell-free DNA and protein blood-based biomarkers yielded an accuracy of only 44% for predicting HCC, likely due to the lack of common recurrent mutations and specific protein markers inherent to HCC (see Cohen J D, et al. Science 2018).

Another challenge in the diagnosis of certain liver diseases by using a non-invasive method is that CECs may be present in two different diseases such that quantitative analysis of CECs may not provide information necessary to distinguish between the two diseases.

To date, there is no non-invasive blood based method available for accurately detecting liver diseases such as HCC, or distinguishing between different liver diseases or between different stages of liver diseases in subjects with chronic liver disease (CLD).

Therefore, there is a need for a non-invasive method for detecting the presence of liver diseases such as HCC and determining stages of liver diseases in CLD patients with high accuracy.

### SUMMARY

The present invention is based, at least in part, on the discovery that hepatic CECs (hCECs) are not exclusive to carcinogenesis, but also can be present in subjects having non-cancer diseases or conditions such as chronic liver disease (CLD). Furthermore, the present invention is based, at least in part, on the discovery that the hCECs in subjects with CLD can be analyzed quantitatively or qualitatively to accurately detect the presence of cancer such as hepatocellular carcinoma (HCC) and/or to accurately characterize the different stages (e.g., early or late stages) of liver diseases or conditions such as liver fibrosis.

In one aspect, the present invention relates to methods of measuring expression levels of hepatocellular carcinoma (HCC) classifier genes in circulating epithelial cells (CECs) of subjects, where the HCC classifier genes include one or more of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

In some embodiments, the HCC classifier genes consist of one or more of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

In some embodiments, the HCC classifier genes consist of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

In some embodiments, the HCC classifier genes also include one, two, three or more additional genes selected from the group consisting of ACTG2, ADM2, AFP, AGR2, ALDH3A1, ALPK3, AMIGO3, ANKRD65, ANLN, AP1M2, ARHGAP11A, ARHGEF39, ASF1B, ASPHD1, AURKA, AXIN2, BAIAP2L2, BEX2, C15orf48, C1orf106, C1QTNF3, C6orf223, CA12, CA9, CAMK2N2, CAP2, CBX2, CCDC170, CCDC28B, CCDC64, CCNE2, CCNF, CD109, CD34, CDC25A, CDC7, CDCA5, CDCA8, CDH13, CDK1, CDKN2A, CDKN2C, CDT1, CELF6, CENPF, CENPH, CENPL, CENPU, CENPW, CKB, CNNM1, COL15A1, COL4A5, COL7A1, COL9A2, CRIP3, CSPG4, CTNND2, CXorf36, CYP17A1, DLK1, DMKN, DSCC1, DTL, DUOX2, ECT2, EEF1A2, EFNA3, EPHB2, EPPK1, ETV4, FABP4, FAM111B, FAM3B, FAM83D, FANCD2, FANCI, FBXL18, FERMT1, FGF19, FLNC, FLVCR1, FOXD2-AS1, FOXM1, FXYD2, GABRE, GAL3ST1, GCNT3, GINS1, GJC1, GMNN, GNAZ, GOLGA2P7, GPC3, GPR64, GPSM1, HRCT1, IGF2BP2, IGSF1, IGSF3, IQGAP3, ITGA2, ITPKA, KIAA0101, KIF11, KIFC1, KIFC2, KNTC1, KRT23, LAMA3, LEF1, LGR5, LINC00152, LINGO1, LPL, LRRC1, LYPD1, MAD2L1, MAGED4, MAGED4B, MAPK12, MAPK8IP2, MAPT, MCM2, MDGA1, MDK, MFAP2, MISP, MKI67, MMP11, MNS1, MPZ, MSC, MSH5, MTMR11, MUC13, MUC5B, MYH4, NAALADL1, NAV3, NCAPG, NDUFA4L2, NEB, NKD1, NMB, NOTCH3, NOTUM, NPM2, NQO1, NRCAM, NT5DC2, NTS, OBSCN, OLFML2A, OLFML2B, PAQR4, PEG10, PI3, PLCE1, PLCH2, PLK1, PLXDC1, PODXL2, POLE2, PPAP2C, PRC1, PTGES, PTGFR, PTHLH, PTK7, PTP4A3, PTTG1, PYCR1, RACGAP1, RBM24, RHBG, RNF157, ROBO1, RP4-800G7.2, RPS6KL1, RRM2, S100A1, SCGN, 5-Sep, SERPINA12, SEZ6L2, SFN, SGOL2, SLC22A11, SLC51B, SLC6A2, SNCG, SOAT2, SP5, SPARCL1, SPINK1, STIL, STK39, SULT1C2, TCF19, TDGF1, THY1, TK1, TMC5, TMEM132A, TMEM150B, TNFRSF19, TNFRSF25, TONSL, TPX2, TRIM16, TRIM16L, TRIM31, TRIM45, TTC39A, UBD, UBE2C, UBE2T, UGT2B11, USH1C, VSIG10L, WDR62, WDR76, and ZWINT.

In one aspect, the present invention relates to methods for detecting the presence of HCC in subjects having chronic liver diseases (CLDs), the method including: (a) measuring expression levels of the HCC classifier genes described herein in CECs of the subjects; and (b) comparing the expression levels of the HCC classifier genes in the CECs of the subject with reference expression levels of HCC classifier genes thereby determining the presence of HCC.

In some embodiments, the expression levels of HCC classifier genes are used to calculate a HCC score, and the calculated HCC score is compared with a reference score, where the presence of HCC is determined based on the presence of a HCC score above the reference score.

In some embodiments, the HCC score is calculated using a random forest analysis.

In some embodiments, the expression levels of HCC classifier genes are compared with the reference expression levels of HCC classifier genes using a multivariate logistic regression modeling approach.

In some embodiments, the expression levels of HCC classifier genes in circulating epithelial cells (CECs) are measured by: (a) obtaining a sample including blood from the subject; (b) removing red blood cells, platelets, and plasma from the sample by size-based exclusion; (c) removing white blood cells (WBCs) from the sample by magnetophoresis; and (d) measuring the expression of a set of genes in the CECs using RNA-sequencing, qRT-PCT, RNA in situ hybridization, protein microarray, or mass spectrometry and protein profiling.

In some embodiments, the HCC being detected is an early stage HCC or a late stage HCC.

In some embodiments, the methods for detecting the precense of HCC in subjects having CLDs also includes: (a) confirming or having confirmed the presence of HCC in the patient by ultrasound imaging, dynamic CT, MRI imaging, needle biopsy, and/or biopsy; and (b) if the presence of HCC in the patient is confirmed, treating or having the subject treated for HCC by surgical removal of the HCC tissue, radiofrequency ablation of the HCC tissue, embolization of the HCC tissue; embolization of HCC tissue, chemotherapy, and/or cryotherapy.

In one aspect, the present invention relates to methods of monitoring subjects having CLD for development of HCC, the method including: (a) detecting the presence of HCC in subjects having CLDs as described herein at an initial time point, and if the HCC score is below the reference score, then (b) performing detection step at one or more subsequent time points. In some embodiments, the detection step is performed at one or more subsequent time points until the presence of HCC is determined. In some embodiments, the initial and each subsequent time point is about three months, six months, or a year apart.

In one aspect, the present invention relates to methods of distinguishing between the presence of early stage liver fibrosis and late stage liver fibrosis in subjects having CLDs, the methods including: (a) detecting concentrations of CECs in blood samples of the subjects; (b) comparing the concentrations of CECs in the blood samples of the subjects with a reference value; (c) diagnosing those subjects that have concentrations of CECs in the blood samples that is below the reference value with early stage fibrosis; and (d) diagnosing those subjects that have concentration of CECs in the blood sample that is above the reference value with late stage fibrosis.

In some embodiments, the subjects have hepatitis B. In some embodiments, the concentrations of CECs are measured by immunofluorescence. In some embodiments, the concentrations of CECs is measured by detecting glypican-3 (GPC3) and/or cytokeratins (CKs).

In one aspect, the present invention relates to methods of monitoring subjects having CLDs for development of advanced fibrosis, the method including: (a) performing a method of distinguishing between the presence of early stage liver fibrosis and late stage liver fibrosis in subjects having CLDs described herein; and if the concentrations of CECs in the blood samples of the subjects are lower than the reference value, then (b) performing the method of distinguishing between the presence of early stage liver fibrosis and late stage liver fibrosis in subjects having CLDs at one or more subsequent time points.

In some embodiments, the method of distinguishing between the presence of early stage liver fibrosis and late stage liver fibrosis in subjects having CLDs is performed at one or more subsequent time points until the subject is diagnosed with late stage fibrosis. In some embodiments, the initial and each subsequent time point is about three months, six months, or a year apart.

In one aspect, the present invention relates to method of monitoring a subject having CLD being treated to prevent the progression of fibrosis or HCC, the method including: (a) performing a method of distinguishing between the presence of early stage liver fibrosis and late stage liver fibrosis in subjects having CLDs, described herein; and if the concentration of CECs in the blood sample of the subject is lower than the reference value, then performing the method of distinguishing between the presence of early stage liver fibrosis and late stage liver fibrosis in subjects having CLDs at one or more subsequent time point; and (b) performing a method of detecting the presence of HCC in subjects having CLDs, described herein, and if the expression levels of the HCC scores are below the reference score, then performing the detection method at one or more subsequent time points.

In some embodiments, the method of distinguishing between the presence of early stage liver fibrosis and late stage liver fibrosis in subjects having CLDs is performed at one or more subsequent time points until the subject is diagnosed with late stage fibrosis, and/or where the method of detecting the presence of HCC in subjects having CLDs is performed at one or more subsequent time points until the presence of HCC is determined. In some embodiments, the first initial and each subsequent time point for performing the method of distinguishing between the presence of early stage liver fibrosis and late stage liver fibrosis in subjects having CLDs or the method of detecting the presence of HCC in subjects having CLDs is about three months, six months, or a year apart, and the second initial and each subsequent time point is about three months, six months, or a year apart.

In some embodiments, the CECs in the subjects' blood are purified or enriched using microfluidic devices. In some embodiments, the microfluidic devices are iChip devices.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In addition, U.S. Patent Application US2016/0312298 A1 is specifically incorporated herein by reference in its entirety, and in some embodiments methods described herein can be used in conjunction with methods described in that application. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a schematic representation of the iChip antigen-agnostic cell sorting device (the iChip device) used to deplete hematopoietic cells. The sample was processed with the iChip device to enrich the sample for CECs, which can be analyzed by immunofluorescence or RNA-Sequencing.

FIG. 2A shows fluorescence microscopy images of immunofluorescence labeled hCECs from peripheral blood of subjects with CLD. Blood samples from patients with HCC or CLD were processed using the iChip device to isolate CECs and stained for DAPI, CD45, glypican-3 (GPC3), and wide-spectrum cytokeratin (CK-WS). A white blood cell (WBC) is shown for comparison.

FIG. 2B is a graph representing detection of immunofluorescence labeled hCECs in the iChip device-processed blood samples from healthy donors (HD) or patients with CLD, HCC, or patients who were treated for HCC with no evidence of malignant disease (HCC NED). P-values were calculated by Mann-Whitney test.

FIG. 2C is a graph representing detection of hCECs in CLD patients with early stage liver fibrosis and patients with advanced fibrosis. P-values were calculated by Mann-Whitney test.

FIG. 3A is a heatmap of the HepG2 gene expression signature obtained from RNA-seq of hCECs in control blood, control blood spiked with 1-50 HepG2 cells, and HepG2 single cell RNA-seq.

FIG. 3B is a heatmap of the liver-specific gene signature obtained from RNA-seq of hCECs from CLD patients, HCC patient, and from flow-sorted WBCs (B, B cells; C, cytotoxic T cells; H, helper T cells; M, monocytes; N, NK cells; G, granulocytes). Heatmap units are represented as log₂ (reads per million+1).

FIG. 3C is a schematic of the random forest algorithm described herein.

FIG. 3D is a graph showing HCC score (vote fraction from the random forest classifier) in CLD, early stage HCC, and late stage HCC. P-values were calculated by Mann-Whitney test.

FIG. 4A is a graph representing detection of glypican-3 positive (GPC3) CECs in the iChip device-processed blood samples from healthy donors (HD) or patients with CLD (CLD), patients with HCC, or patients who have previously had HCC but do not show evidence of malignant disease after being treated for HCC (HCC NED). P-values were calculated by Mann-Whitney test.

FIG. 4B is a graph representing detection of CECs expressing wide spectrum cytokeratin (CK+ cells) in the iChip device-processed blood samples from healthy donors (HD) or patients with CLD (CLD), patients with HCC (HCC), or patients who have previously had HCC but do not show evidence of malignant disease after being treated for HCC (HCC NED). P-values were calculated by Mann-Whitney test.

FIG. 4C is a graph representing detection of hCEC (cells that are CK+ or GPC3+) in HBV CLD patients (without HCC) stratified by fibrosis stage (with early stage defined as F1 or F2 and advanced fibrosis defined as F3 or F4). P-values were calculated by Mann-Whitney test.

FIG. 4D is a graph representing CEC concentration in CLD patients stratified by etiology of liver disease: non-alcoholic steatohepatitis (NASH); hepatitis B virus (HBV); hepatitis C virus (HCV); autoimmune hepatitis (AIH); primary sclerosing cholangitis (PSC). P-values were calculated by Mann-Whitney test.

FIG. 5A is a graph representing HCC score (vote fraction from the random forest classifier) of CECs in CLD patients, HCC patients who received treatment but still had active disease at the time of blood draw (HCC On Tx), and patients with active HCC who were treatment-naïve (HCC No Tx). P-values shown were calculated by Mann-Whitney test.

FIG. 5B is a graph representing receiver operating characteristic (ROC) curve for the HCC classifier created by multivariable logistic regression modeling.

FIG. 5C is a graph representing ROC curve for the HCC random forest classifier.

### DETAILED DESCRIPTION

The present invention is based, at least in part, on the discovery that hCECs are not exclusive to carcinogenesis, but also can be present in subjects having non-cancer diseases or conditions such as chronic liver disease (CLD). Furthermore, the present invention is based, at least in part, on the discovery that the hCECs in subjects with CLD can be analyzed quantitatively or qualitatively to accurately detect the presence of cancer such as hepatocellular carcinoma (HCC) and/or to accurately characterize the stage (e.g., early or late stage) of a liver disease or liver condition such as liver fibrosis.

As demonstrated herein, cells from diseased livers circulating in the bloodstream (i.e., hCECs) are detected both quantitatively (e.g., by immunofluorescence) and qualitatively (e.g., gene expression profile or expression levels of HCC classifier genes) for use in diagnosis of HCC and CLD. Important applications of this liquid biopsy include detection or diagnosis of a liver disease or condition such as HCC, CLD etiology determination, liver fibrosis staging, and HCC surveillance or monitoring. The present invention can be applied to both diagnosis and monitoring of patients with liver conditions such as CLDs.

As used herein, the phrases “accurately diagnose” and “accurately detect” with respect to a disease or a condition refer to predicting the presence of the disease or the condition with a high degree of sensitivity (i.e., true positive rate or detecting a disease or a condition when the disease or the condition is present) or a high degree of specificity (i.e., true negative rate or not detecting a disease or a condition when the disease or the condition is not present). In some embodiments, the phrases “accurately diagnose” and “accurately detect” can also mean being able to detect the presence of a disease or a condition with a true positive rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%. In some embodiments, the phrases “accurately diagnose” and “accurately detect” can mean being able to detect the presence of a disease or a condition with a true negative rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%.

As used herein, the phrase “accurately distinguish” with respect to two diseases or conditions, can refer to detecting the presence of a first disease or a first condition with a high degree of sensitivity (i.e., detecting a first disease or condition when the first disease or condition is present, i.e., true positive rate) or a high degree of specificity (i.e., not detecting a first disease or condition when the first disease or condition is not present, i.e., true negative rate), regardless of whether the second disease or condition is also present or absent. In some embodiments, the phrase “accurately distinguish” can mean being able to detect the presence of a disease or a condition with a true positive rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%. In some embodiments, the phrase “accurately distinguish” can mean being able to detect the presence of a disease or a condition with a true negative rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%.

As used herein, the phrase “accurately distinguish” with respect to different stages of a disease or a condition can refer to detecting the presence of a particular stage of the disease (e.g., advanced fibrosis in liver) with a high degree of sensitivity (i.e., detecting the stage of a disease or condition when the disease or condition is present at that stage, i.e., true positive rate) or a high degree of specificity (i.e., not detecting a stage of a disease or condition when the disease or condition is not present at that stage, i.e., true negative rate) so that the particular stage of the condition or disease can be predicted. In some embodiments, the phrase “accurately distinguish” can mean being able to detect the presence of a stage of a disease or a condition with a true positive rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%. In some embodiments, the phrase “accurately diagnose” can mean being able to detect the presence of a disease or a condition with a true negative rate of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, and at least about 99.9%.

As used herein, the term “circulating epithelial cells (CECs)” can refer to cells of epithelial origin that are shed from a tissue (e.g., diseased tissue, tumor tissue, or non-tumor tissue) and present in the blood, i.e. in circulation. Cell markers (e.g. marker genes) that can be used to identify and/or isolate CECs from other components of the blood are described below herein. In some embodiments, the CECs from a subject with a liver disease (e.g., HCC and/or CLD) are predominantly hepatic CECs (hCECs), for example, as determined by immunofluorescence staining of the CECs with genes expressed in hepatocytes (e.g., GPC3 and CKs).

As used herein, the term “chronic liver disease (CLD)” refers to a disease process of the liver involving progressive destruction and regeneration of the liver parenchyma. In some embodiments, CLD can lead to fibrosis cirrhosis. In some other embodiments, CLD can result in complications such as portal hypertension (e.g., ascites, hyperplenism, and lower esophageal varices and rectal varices) hepatopulmonary syndrome, hepatorenal syndrome, encephalopathy, or HCC. CLD can also refer to disease of the liver which lasts over a period of six months, one year, two years, three years, four years, five years, or more than five years. CLD can be caused by hepatitis B, hepatitis C, cytomegalovirus, Epstein Bar virus, yellow fever viruses, alcoholic liver disease, and/or drug induced liver disease from methotrexate, amiodarone, nitrofurantoin, or acetaminophen. In other embodiments, CLD can be caused by non-alcoholic fatty liver disease, haemochromatosis, Wilson's disease, or autoimmune responses such as primary biliary cholangitis or primary sclerosing cholangitis.

As used herein, the term “monitoring” or “surveillance” refers to periodically assessing a subject or a patient (e.g., a subject who is at risk of developing a condition) for the presence of a disease or a condition. In some embodiments, the periodic assessment can occur about every day, about every other day, about once a week, about once every other week, about every month, about every 2 months, about every 3 months, about every 4 months, about every 5 months, about every 6 months, about every 7 months, about every 8 months, about every 9 months, about every year, about every 18 months, about every 2 years, about every 3 years, about every 4 years, about every 5 years, about every 6 years, about every 7 years, about every 8 years, about every 9 years, or about every 10 years. This recurring assessment of a subject or a patient for the presence of a disease or a condition can continue until (1) the disease or the condition is detected in the subject or the patient; (2) the patient is no longer at risk of developing the disease or the condition; (3) at the discretion of the subject receiving the monitoring or the person administering the monitoring; or (4) discontinuation of the recurring assessment is necessary due to other reasons. The interval with which a subject is assessed for the presence of a disease or a condition can be adjusted during the course of the monitoring.

As used herein the term “ensemble learning method” refers to a supervised learning algorithm such as random forest that can be trained and then used to make predictions.

As used herein the term “hepatocellular carcinoma (HCC)” refers to a type of primary liver cancer prevalent in subjects with CLD. HCC can develop in patients with underlying cirrhotic liver disease of various etiologies, including patients with negative markers for HBV infection and who have HBV DNA integrated in the hepatocyte genome. Epidemiology, etiology, and carcinogenesis of HCC has been described in Ghouri Y A, et al., J Carcinog 2017; 16:1, which is incorporated by reference herein.

As used herein, the phrase “early stage HCC” can refer to HCC being within the Milan criteria. As used herein, the phrase “late stage HCC” can refer to HCC being outside of the Milan criteria. Milan criteria requires the subject with HCC meet the following criteria: HCC being one lesion smaller than 5 cm or up to 3 lesions, each smaller than 3 cm; no extrahepatic manifestations; and no evidence of gross vascular invasion. In other words, “early stage HCC” meets all Milan criteria and “late stage HCC” does not meet all Milan criteria.

As used herein, the term “early stage liver fibrosis” and “late stage liver fibrosis” refer to F1 or F2 stages, and F3 or F4 stages, respectively, as defined by the METAVIR classification.

The methods described herein can be used to accurately diagnose or predict the presence of cancer, e.g., HCC, in a patient with a non-cancerous disease condition, e.g., CLD, by detecting and analyzing expression of a set of genes in the CECs of the patient using a classifier that is based on an ensemble learning method such as random forest classifier.

In some embodiments, hCECs from subjects with CLD (e.g., subjects with Hepatitis B or subjects who are infected with Hepatitis B Virus) can be analyzed (e.g., qualitatively) to accurately distinguish between subjects with and without HCC. In other embodiments, hCECs from subjects with CLD can be quantitatively measured to accurately distinguish between subjects with early stage liver fibrosis and subjects with late stages liver fibrosis.

As demonstrated herein, the presence of cancer, e.g., HCC, and the presence of non-cancer diseases or conditions, e.g., CLD, are associated with the increased presence of CECs. The increased presence of CECs is also associated with the previous presence of cancer (e.g., HCC) which was treated to result in no clinical evidence of disease (e.g., in HCC patients who underwent curative treatment and had no clinical evidence of the disease).

Thus the methods can include the detection and analysis of a set of genes (e.g., HCC classifier genes) using a variety of statistical and computational prediction method (e.g., an ensemble learning method such as random forest classifier or a statistical method such as multivariable logistic regression), to detect the presence of a cancer, e.g., HCC.

The method can, in some embodiments, detect the presence of cancer at an early stage, which may otherwise be difficult to detect using a currently known method such as ultrasound imaging, dynamic CT, MRI imaging, needle biopsy, or biopsy.

In some embodiments, microfluidic (e.g., “lab-on-a-chip” or the iChip device) can be used in the present methods to separate, purify, enrich, or prepare CECs. Such devices have been successfully used for microfluidic flow cytometry, continuous size-based separation, chromatographic, or magnetophoretic separation. For Example, the iChip device and various other embodiments of such devices are described in U.S. Patent Application US2016/0312298 A1 (which is incorporated herein by reference) can be used for separating hCECs from a mixture of cells, or preparing an enriched population of hCECs. In particular, such devices can be used for the isolation of hCECs from complex mixtures such as whole blood.

In some embodiments, the devices retain at least 75%, e.g., 80%, 90%, 95%, 98%, or 99% of the desired cells compared to the initial sample mixture, while enriching the population of desired cells by a factor of at least 100, e.g., by 1000, 10,000, 100,000, or even 1,000,000 relative to one or more non-desired cell types. In one example, a detection module can be in fluid communication with a separation or enrichment device. The detection module can operate using any method of detection disclosed herein, or other methods known in the art. For example, the detection module includes a microscope, a cell counter, a magnet, a biocavity laser (see, e.g., Gourley et al., J. Phys. D: Appl. Phys., 36: R228-R239 (2003)), a mass spectrometer, a PCR device, an RT-PCR device, a microarray, a device for performing RNA in situ hybridization, or a hyperspectral imaging system (see, e.g., Vo-Dinh et al., IEEE Eng. Med. Biol. Mag., 23:40-49 (2004)). In some embodiments, a computer terminal can be connected to the detection module. For instance, the detection module can detect a label that selectively binds to cells, proteins, or nucleic acids of interest, e.g., transcripts of HCC classifier genes or encoded proteins.

In some embodiments, the microfluidic system includes (i) a device for separation or enrichment of CECs (e.g., hCECs); (ii) a device for lysis of the enriched CECs; and (iii) a device for detection of gene transcripts (e.g., transcripts of HCC classifier genes) or encoded proteins.

In some embodiments, a population of CECs prepared using a microfluidic device as described herein is used for analysis of expression of gene transcripts or proteins using known molecular biological techniques, e.g., as described above and in Sambrook, Molecular Cloning: A Laboratory Manual, Third Edition (Cold Spring Harbor Laboratory Press; 3rd edition (Jan. 15, 2001)); and Short Protocols in Molecular Biology, Ausubel et al., eds. (Current Protocols; 52 edition (Nov. 5, 2002)).

In general, devices for detection and/or quantification of expression of classifier genes useful for cancer diagnosis or encoded proteins in an enriched population of CECs (e.g., CTCs) are described herein and can be used for the early detection of cancer, e.g., tumors of epithelial origin, e.g., early detection of liver, pancreatic, lung, breast, prostate, renal, ovarian or colon cancer.

As described herein, the phrase “differential expression analysis” can refer to performing computational or statistical analysis on expression level of individual genes (e.g., individual HCC classifier genes) and/or expression patterns of multiple genes (e.g., multiple HCC classifier genes) in a sample (e.g., cell, e.g., CEC, e.g., hCEC). The term “differential expression” can mean over-expression (expressing a gene at a higher level than the reference value) or under-expression (expressing a gene at a lower level than the reference value). In some embodiments, a differential expression analysis can compare the expression levels or patterns in a sample with a reference value (e.g., expression levels or patterns of one or more genes in a sample from a non-diseased counterpart cell or tissue). In other embodiments, the expression levels or patterns can be normalized to expression levels of one or more control genes, or may be quantified in a non-relative manner (e.g., transcript copies per volume or absolute copy number). The gene expression levels can be measured by any of the known methods, such RNA-sequencing, qRT-PCT, RNA in situ hybridization, protein microarray, and/or mass spectrometry and protein profiling. Other known biochemical, or molecular biology techniques can be used to detect the expression of genes. In some embodiments, RNA-sequencing and qRT-PCT is the preferred method for measuring gene expression levels.

The differential expression analysis can be performed by any one of the known statistical or computational methods, for example, an ensemble learning method such as random forest classifier or a statistical method such as multivariable logistic regression.

In one aspect, the present invention provides methods including measuring expression levels of hepatocellular carcinoma (HCC) classifier genes in circulating epithelial cells (CECs) of a subject. The overexpression of HHC classifier genes by the CECs of subjects was determined as being highly predictive of the presence of HCC in the subjects (see e.g., Example 1-4). In some embodiment, the HCC classifier genes include one, two, three, or more of (e.g., all of) TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32. In some embodiments, the HCC classifier genes can be include all of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1. In other embodiments, the HCC classifier genes can also include one or more other genes that are overexpressed in HCC, e.g., one or more of ACTG2, ADM2, AFP, AGR2, ALDH3A1, ALPK3, AMIGO3, ANKRD65, ANLN, AP1M2, ARHGAP11A, ARHGEF39, ASF1B, ASPHD1, AURKA, AXIN2, BAIAP2L2, BEX2, C15orf48, C1orf106, C1QTNF3, C6orf223, CA12, CA9, CAMK2N2, CAP2, CBX2, CCDC170, CCDC28B, CCDC64, CCNE2, CCNF, CD109, CD34, CDC25A, CDC7, CDCA5, CDCA8, CDH13, CDK1, CDKN2A, CDKN2C, CDT1, CELF6, CENPF, CENPH, CENPL, CENPU, CENPW, CKB, CNNM1, COL15A1, COL4A5, COL7A1, COL9A2, CRIP3, CSPG4, CTNND2, CXorf36, CYP17A1, DLK1, DMKN, DSCC1, DTL, DUOX2, ECT2, EEF1A2, EFNA3, EPHB2, EPPK1, ETV4, FABP4, FAM111B, FAM3B, FAM83D, FANCD2, FANCI, FBXL18, FERMT1, FGF19, FLNC, FLVCR1, FOXD2-AS1, FOXM1, FXYD2, GABRE, GAL3ST1, GCNT3, GINS1, GJC1, GMNN, GNAZ, GOLGA2P7, GPC3, GPR64, GPSM1, HRCT1, IGF2BP2, IGSF1, IGSF3, IQGAP3, ITGA2, ITPKA, KIAA0101, KIF11, KIFC1, KIFC2, KNTC1, KRT23, LAMA3, LEF1, LGR5, LINC00152, LINGO1, LPL, LRRC1, LYPD1, MAD2L1, MAGED4, MAGED4B, MAPK12, MAPK8IP2, MAPT, MCM2, MDGA1, MDK, MFAP2, MISP, MKI67, MMP11, MNS1, MPZ, MSC, MSH5, MTMR11, MUC13, MUC5B, MYH4, NAALADL1, NAV3, NCAPG, NDUFA4L2, NEB, NKD1, NMB, NOTCH3, NOTUM, NPM2, NQO1, NRCAM, NT5DC2, NTS, OBSCN, OLFML2A, OLFML2B, PAQR4, PEG10, PI3, PLCE1, PLCH2, PLK1, PLXDC1, PODXL2, POLE2, PPAP2C, PRC1, PTGES, PTGFR, PTHLH, PTK7, PTP4A3, PTTG1, PYCR1, RACGAP1, RBM24, RHBG, RNF157, ROBO1, RP4-800G7.2, RPS6KL1, RRM2, S100A1, SCGN, 5-Sep, SERPINA12, SEZ6L2, SFN, SGOL2, SLC22A11, SLC51B, SLC6A2, SNCG, SOAT2, SP5, SPARCL1, SPINK1, STIL, STK39, SULT1C2, TCF19, TDGF1, THY1, TK1, TMC5, TMEM132A, TMEM150B, TNFRSF19, TNFRSF25, TONSL, TPX2, TRIM16, TRIM16L, TRIM31, TRIM45, TTC39A, UBD, UBE2C, UBE2T, UGT2B11, USH1C, VSIG10L, WDR62, WDR76, and ZWINT.

In another aspect, the present invention provides methods for detecting the presence of HCC in subjects having a chronic liver disease (CLD). The methods can include: (a) measuring expression levels of HCC classifier genes in CECs of a subject; and (b) comparing the expression levels of HCC classifier genes in the CECs of the subject with reference expression levels of HCC classifier genes thereby determining the presence of HCC.

In another aspect, the present invention provides methods of monitoring subjects having CLD for development of HCC. The methods can include: (a) measuring expression levels of HCC classifier genes in CECs of a subject and comparing the expression levels of HCC classifier genes in the CECs of the subject with reference expression levels of HCC classifier genes at an initial time point; and if the expression levels of the HCC classifier genes are below the reference level, then (b) performing the step again at a subsequent time point, and optionally at additional time points, e.g., until the expression levels of HCC classifier genes are above the reference level. This assessment can be formed by first calculating a HCC score (e.g., the vote fraction from the RF classifier) or other metrics that indicate the degree of differential expression of HCC classifier genes in the subject's CECs, as compared to a reference score, or other reference metrics values.

In another aspect, the present invention provides methods of distinguishing between the presence of early stage liver fibrosis and late stage liver fibrosis in a subject having CLD. The methods can include: (a) detecting a concentration of CECs in a blood sample of a subject; (b) comparing the concentration of CECs in the blood sample of the subject with a reference value; (c) diagnosing the subject with early stage fibrosis if the subject's blood concentration of CECs is below the reference value; and (d) diagnosing the subject with late stage fibrosis if the subject's blood concentration of CECs is above the reference value.

In another aspect, the present invention provides methods of monitoring a subject having CLD for development of advanced fibrosis. The methods can include: (a) detecting a concentration of CECs in a blood sample of a subject and comparing the blood CEC concentration to a reference value; and if the concentration of CECs in the blood sample of the subject is lower than the reference value, then (b) performing the same detection and comparison step at one or more subsequent time points, e.g., until the concentration of CECs in the blood sample of the subject is higher than the reference value.

In some embodiments, the expression levels of HCC classifier genes are used to calculate a HCC score, preferably using a random forest analysis, and the method includes comparing the HCC score with a reference score, wherein the presence of HCC is determined based on the presence of a HCC score above the reference score.

In some embodiments, the expression levels of HCC classifier genes are compared with the reference expression levels of HCC classifier genes using a multivariate logistic regression modeling approach.

In some embodiments, the expression levels of HCC classifier genes in circulating epithelial cells (CECs) are measured by: (a) obtaining a sample comprising blood from the subject; (b) removing red blood cells, platelets, and plasma from the sample by size-based exclusion; (c) removing white blood cells (WBCs) from the sample by magnetophoresis; and (d) measuring the expression of a set of genes in the CECs using RNA-sequencing, qRT-PCT, RNA in situ hybridization, protein microarray, or mass spectrometry and protein profiling.

In some embodiments, the HCC being detected is an early stage HCC or a late stage HCC.

In some embodiments, the method also includes (a) confirming or having confirmed the presence of HCC in the patient by ultrasound imaging, dynamic CT, MIR imaging, needle biopsy, and/or biopsy; and (b) if the presence of HCC in the patient is confirmed, treating or having the subject treated for HCC by surgical removal of the HCC tissue, radiofrequency ablation of the HCC tissue, embolization of the HCC tissue; embolization of HCC tissue, chemotherapy, and/or cryotherapy.

In some embodiments, the initial and each subsequent time point for measuring and comparing the blood CEC concentration or for measuring and comparing HCC classifier gene is about three months, six months, or a year apart. In some embodiments, the subject has hepatitis B or not have hepatitis B. In some embodiments, the concentration of CECs is measured by immunofluorescence. In some embodiments, the concentration of CECs is measured by detecting glypican-3 (GPC3) and/or cytokeratins (CKs).

### Diagnosis and Treatment of Liver Diseases

Once a liver disease such as CLD or HCC are detected in a subject, the presence of the disease such as CLD or HCC may be confirmed using other methods.

Diagnosis or Detection of HCC

HCC can be further confirmed or diagnosed by analyzing blood sample using traditional methods, including a complete blood count (CBC), electrolytes, liver function tests (LFTs), coagulation studies (e.g., international normalized ratio (INR) and partial thromboplastin time (PTT)), and alpha-fetoprotein (AFP) determination.

Various imaging techniques can be used to diagnose HCC. For example, ultrasonography offers a relatively inexpensive method of screening without the cost of magnetic resonance imaging (MRI) or the exposure to radiation and potentially nephrotoxic contrast agents required for computed tomography (CT). Ultrasonography as a screening method is reported to have 60% sensitivity and 97% specificity in the cirrhotic population, and it has been demonstrated to be cost-effective. Due to this low-sensitivity, findings on ultrasound examination should be confirmed with further imaging studies and potentially biopsy.

HCC can be detected using CT imaging, preferably with early enhancement on the arterial phase with rapid washout of contrast on the portal venous phase of a three-phase contrast scan. HCC can also be detected using MRI.

HCC can be detected by biopsy, especially for subjects with HCCs that are larger than 2 cm with low levels of alpha-fetoprotein or in whom ablative treatment or transplant is contraindicated.

In patients with elevated AFP and consistent imaging characteristics, patients can be treated presumptively for HCC without a biopsy. Patients preferably can also undergo evaluation for extrahepatic disease (primarily pulmonary metastasis) with cross-sectional imaging; this would preclude curative locoregional therapy

### Treatment of HCC

HCC can be treated using a number of methods known in the art, including by liver transplantation-however a limited supply of donor organs limit the availability of transplantation as an option for many subjects. HCC can also be treated using resection, radiofrequency ablation (RFA). Systemic therapy with sorafenib (or, if sorafenib fails, with regorafenib, nivolumab, or lenvatinib), can be used to bridge patients to transplant or to delay recurrence of HCC. In patients who experience a recurrence following resection or transplantation, aggressive surgical treatment appears to be associated with the best possible outcome.

HCC can be treated by transcatheter arterial chemoembolization, which selectively cannulates the feeding artery to the tumor and delivers high local doses of chemotherapy, including doxorubicin, cisplatin, or mitomycin C. To prevent systemic toxicity, the feeding artery is occluded with gel foam or coils to prevent flow.

HCC can be treated by chemotherapy-however, HCC is minimally responsive to systemic chemotherapy. For example, doxorubicin-based regimens, which appears to have the greatest efficacy, has a response rates of 20-30% and a minimal impact on survival.

For patients with Child class C cirrhosis and contraindications for transplantation, HCC can be managed by focusing on pain control, ascites, edema, and portosystemic encephalopathy management.

HCC can be treated surgically. Presently, in view of the absence of effective chemotherapy and the insensitivity of HCC to radiotherapy, complete tumor extirpation is the only option for a long-term cure. Resection of the tumor by partial hepatectomy can be accomplished in a limited number of patients (generally <15-30%) due to the degree of underlying cirrhosis.

### Diagnosis and Treatment of Liver Cirrhosis

Chronic liver disease can include liver cirrhosis, which is characterized by fibrosis and the conversion of normal liver architecture into structurally abnormal nodules. The progression of liver injury to cirrhosis may occur over weeks to years. In addition to fibrosis, the complications of cirrhosis include, but are not limited to, portal hypertension, ascites, hepatorenal syndrome, and hepatic encephalopathy.

Liver cirrhosis can occur in Hepatitis C alcoholic liver disease, NASH; and Hepatitis B. Hepatic fibrosis can occur due to alteration in the normally balanced processes of extracellular matrix production and degradation in liver. In liver cirrhosis, stellate cells can become activated into collagen-forming cells by a variety of paracrine factors. Such factors can be released by hepatocytes, Kupffer cells, and sinusoidal endothelium following liver injury. For example, increased levels of the cytokine transforming growth factor beta1 (TGF-beta1) are observed in patients with chronic hepatitis C and those with cirrhosis. TGF-beta1, in turn, stimulates activated stellate cells to produce type I collagen.

Diagnosis of Liver Cirrhosis

Severity of liver cirrhosis is commonly assessed using the Child-Turcotte-Pugh (CTP) system, a scoring system for assessing the severity of cirrhosis by considering the clinical variables encephalopathy, presence and/or severity of ascites, levels of bilirubin and albumin levels in blood, and prothrombin time.

Severity of liver cirrhosis can also be assessed using the Model for End-Stage Liver Disease (MELD) scoring system, by considering the clinical variables of number of times dialysis was needed, blood levels of creatinine, bilirubin levels, sodium, and prothrombin time.

Treatment of Liver Cirrhosis

Subjects with severe CLD (e.g., decompensated cirrhosis) can be treated using liver transplantation. Liver transplantation has a 1-year survival rate of 85-90% and a 5-year survival rate of higher than 70%. Quality of life after liver transplant is good or excellent in most cases. However, a limited supply of donor organs limit the availability of transplantation as an option for many subjects.

A number of therapies are available to prevent or delay the development of cirrhosis in subjects with CLD: prednisone and azathioprine for treating autoimmune hepatitis, interferon and other antiviral agents for treating hepatitis B and C, phlebotomy for hemochromatosis, ursodeoxycholic acid for primary biliary cirrhosis, and trientine and zinc for Wilson disease. NASH is an advanced form of nonalcoholic fatty liver disease (NAFLD), which are being evaluated for treatment using allosteric Acetyl-CoA Carboxylase (ACC) inhibitors (e.g., NDI-010976/GS-0976), obeticholic acid, thiazolidinediones (e.g., pioglitazone, rosiglitazone, lobeglitazone, ciglitazone, darglitazone, englitazone, netoglitazone, rivoglitazone, troglitazone, balaglitazone), elafibranor (GFT505), obeticholic acid (OCA), apoptosis signal-regulating kinase 1 (ASK1) inhibitor (selonsertib), dual CCR2/CCR5 inhibitor cenicriviroc (CVC, also TBR-652 or TAK-652), and vitamin E.

These therapies are less effective if chronic liver disease evolves into cirrhosis. Once cirrhosis develops, treatment is aimed at the management of complications arising from cirrhosis. For example, cirrhosis-related zinc deficiency can be treated with zinc sulfate at 220 mg orally twice daily to improve dysgeusia and to stimulate appetite. Furthermore, zinc is effective in the treatment of muscle cramps and is adjunctive therapy for hepatic encephalopathy. Pruritus in subjects with CLD (e.g., cholestatic liver diseases or Hepatitis C) can be treated with Cholestyramine, antihistamines (eg, diphenhydramine, hydroxyzine) and ammonium lactate 12% skin cream (Lac-Hydrin), include ursodeoxycholic acid, doxepin, and rifampin. Naltrexone may be effective but is often poorly tolerated. Gabapentin is an unreliable therapy. Patients with severe pruritus may require institution of ultraviolet light therapy or plasmapheresis. Hypogonadism in male subjects with CLD can be treated with topical testosterone preparations. Osteoporosis in subjects with CLD (especially chronic cholestasis or primary biliary cirrhosis) can be treated with calcium and vitamin D supplements. In addition, patients with CLD can be vaccinated against hepatitis A.

### Examples

The invention is further described in the following examples, which do not limit the scope of the invention described in the claims.

### Methods

The following materials and methods were used in the Examples set forth below.

### Clinical Protocol

Patient medical data were collected from patient electronic medical record with patient permission and a maximum of 20 ml of blood was obtained from patients at any given blood draw in two 10-mL EDTA tubes, and approximately 8-15 ml of blood was processed per patient.

Microfluidic Purification of CECs from Whole Blood Using the iChip Device

Biotinylated primary antibodies against anti-human CD45 antibody (clone 2D1, R&D Systems, BAM1430) and anti-human CD66b antibody (Abd Serotec, 80H3) were spiked into whole blood (5-10 mL total volume) at 100 fg/WBC and 37.5 fg/WBC, respectively, and incubated rocking at room temperature for 20 min. Dynabeads MyOne Strepavidin T1 (Life Technologies, 65602) magnetic beads were then added and incubated rocking at room temperature for an additional 20 min. The total blood volume (5-10 mL) was then run on the iChip device as previously described.⁸

### Immunofluorescent Staining of CECs

Cells in an aliquot of the iChip device-processed blood samples were fixed with 2% paraformaldehyde for 10 min and then applied to glass slides via cytospin using a Shandon EZ Megafunnel (ThermoFisher A78710001) at 2000 rpm for 5 min. Slides were washed with PBS and blocked with 5% donkey serum+0.3% Triton-X in PBS for 1 hr at room temperature (RT). Primary antibodies (each at 1:50 dilution in PBS, 0.1% BSA, 0.3% Triton-X) against wide spectrum cytokeratin (WS CK, Abcam ab9377), glypican-3 (Abcam ab81263), and CD45 (Becton Dickenson 555480) were then added and incubated for 1 hr at RT. Secondary antibodies (each at 1:200 dilution in PBS, 0.1% BSA, 0.3% Triton-X) directed against each of the primary antibodies were then used for fluorescent labelling, incubated for 1 hr at RT protected from light: 1) cytokeratin—donkey anti-rabbit Alexa-647 (Jackson ImmunoResearch 711-605-152); 2) glypican-3—donkey anti-sheep Cy3 (Jackson ImmunoResearch 713-165-003); 3) CD45—donkey anti-mouse Alexa-488 (Jackson ImmunoResearch 715-545-150), which were. Cell nuclei were counterstained with DAPI (5 μg/mL in PBS, Life Technologies). Slides were mounted using ProLong Gold Antifade Reagent (Life Technologies). Stained cells were imaged by fluorescence microscopy (TiE or Eclipse 90i, Nikon) using the appropriate filter cubes for image acquisition and the BioView platform for automated image analysis. All candidate CECs detected were reviewed and scored based on intact morphology, localization of CEC markers (WS CK Alexa-647 and/or GPC3 Cy3) with DAPI nuclear counterstain, and absence of leukocyte markers (CD45 Alexa-488).

### HepG2 Cell Spike-in

HepG2 cells were cultured following American Type Culture Collection-recommended culturing conditions. Individual cells were micropipetted using an Eppendorf TransferMan NK2 micromanipulator and introduced into 4 mL of blood from healthy donors, before processing through the iChip device.

### RNA-Sequencing of CECs

The iChip device-processed blood sample aliquot was pelleted and flash frozen in RNAlater (Thermo-Fisher Scientific) at −80 deg C. RNA was extracted (RNEasy Micro, Qiagen) and processed as follows for RNA-seq. Amplified cDNA was generated from RNA from each sample using the SMARTer Ultra Low Input RNA Kit (v3 or v4) for Sequencing (Clontech Laboratories) according to the manufacturer's protocol. Briefly, 1 μl of a 1:50,000 dilution of ERCC RNA Spike-In Mix (Life Technologies) was added to each sample. First-strand synthesis of RNA molecules was performed using the poly-dT-based 3′-SMART CDS primer II A followed by extension and template switching by the reverse transcriptase. The second strand synthesis and amplification PCR were run for 18 cycles, and the amplified cDNA was purified with a 1× Agencourt AMPure XP bead cleanup (Beckman Coulter). The Nextera® XT DNA Library Preparation kit (Illumina) was used for sample barcoding and fragmentation according to the manufacturer's protocol. 1 ng of amplified cDNA was used for the enzymatic tagmentation followed by 12 cycles of amplification and unique dual-index barcoding of individual libraries. PCR product was purified with a 1.8× Agencourt AMPure XP bead cleanup. The eluted cDNA libraries did not undergo the bead-based library normalization step in the Nextera XT protocol. Library validation and quantification was performed by quantitative PCR using the KAPA SYBR® FAST Universal qPCR Kit (Kapa Biosystems). The individual libraries were pooled at equal concentrations, and the pool concentration was determined using the KAPA SYBR® FAST Universal qPCR Kit. The pool of libraries was subsequently sequenced in three replicates on a HiSeq 2500 in Rapid Run Mode using a 2×100 base pair kit and a dual flow cell. The paired-end reads from the three sequencing runs were combined and aligned to the hg38 genome from http://genome.ucsc.edu using the STAR v2.4.0h aligner with default settings. Reads that did not map or mapped to multiple locations were discarded. Duplicate reads were marked using the MarkDuplicates tool in picard-tools-1.8.4 and were removed. The uniquely aligned reads were counted using htseq-count in the intersection-strict mode against the publicly available Homo_sapiens.GRCh38.79.gtf annotation table. Data were then imported into the R statistical programming language for analysis. All RNA-seq raw data has been submitted to NCBI GEO: accession GSE117623.

### Flow Sorting of White Blood Cells

For a subset of HCC patients, the iChip device-processed blood sample was divided into two equal aliquots: one aliquot was pelleted and flash frozen as above; the second was flow sorted to isolate subtypes of contaminating white blood cells (monocytes, granulocytes, NK cells, cytotoxic T cells, helper T cells, and B cells). Cells were fixed with Cytofix (BD Biosciences 554655). The following antibodies were used: CD45 (Beckman Coulter IM0782U), CD56 (Beckman Coulter IM2073U), CD16 (Biolegend 360712), CD14 (Biolegend 301808), CD3 (Biolegend 317330), CD19 (Biolegend 302216), CD4 (Biolegend 300556), CD8 (Biolegend 301016), CD66b (Biolegend 305112). As described above, flow sorted cells were pelleted, flash frozen in RNAlater, and subjected to RNA-seq.

### Example 1: Overview of Classification of CLD Patients Using Random Forest Classifier

The RNA-seq raw data consisted of read counts for 59,074 transcripts on 64 CLD and 52 HCC samples. Of those, only samples with more than 250k total reads were kept, leaving 44 CLD and 39 HCC samples. In order to narrow the list of features in our data set to those with a higher likelihood of relevance for predicting HCC status, RNA-seq expression data was obtained from The Cancer Genome Atlas (TCGA) liver cancer project (LIHC), which contains expression counts for both normal liver and HCC tissue. A differential expression analysis was performed on this data set to identify transcripts overexpressed in HCC vs. normal liver tissue using the DESeq2 package (version 1.16.1) with Benjamini-Hochberg correction for multiple hypothesis testing in R. Using this analysis combined with RNA-seq data on bulk white blood cell (WBC) subsets obtained via flow sorting, a list of transcripts with adjusted p-value <0.05, log 2 fold change >2, WBCs <50 rpm in the summed WBC subsets, and a mean expression in healthy liver tissue >0.5 rpm was constructed. This list was used to narrow the 59,074 features in the raw data set to a set of 248 transcripts more likely to be predictive of HCC. The set of 248 transcripts were: ACTG2, ADM2, AFP, AGR2, AKR1B10, ALDH3A1, ALPK3, AMIGO3, ANKRD65, ANLN, AP1M2, APOBEC3B, ARHGAP11A, ARHGEF39, ASF1B, ASPHD1, ASPM, AURKA, AXIN2, BAIAP2L2, BEX2, C15orf48, C1orf106, C1QTNF3, C6orf223, CA12, CA9, CAMK2N2, CAP2, CBX2, CCDC170, CCDC28B, CCDC64, CCNA2, CCNB1, CCNE2, CCNF, CD109, CD34, CDC20, CDC25A, CDC6, CDC7, CDCA5, CDCA8, CDH13, CDK1, CDKN2A, CDKN2C, CDT1, CELF6, CENPF, CENPH, CENPL, CENPU, CENPW, CKB, CNNM1, COL15A1, COL4A5, COL7A1, COL9A2, CRIP3, CSPG4, CTNND2, CXorf36, CYP17A1, DLK1, DMKN, DSCC1, DTL, DUOX2, E2F1, ECT2, EEF1A2, EFNA3, EPHB2, EPPK1, ETV4, EZH2, F2RL3, FABP4, FAM111B, FAM3B, FAM83D, FANCD2, FANCI, FBXL18, FBXO32, FERMT1, FGF19, FLNC, FLVCR1, FMO1, FOXD2-AS1, FOXM1, FXYD2, GABRE, GAL3ST1, GCNT3, GINS1, GJC1, GMNN, GNAZ, GOLGA2P7, GPC3, GPR64, GPSM1, HRCT1, IGF2BP2, IGSF1, IGSF3, IQGAP3, ITGA2, ITPKA, KIAA0101, KIF11, KIFC1, KIFC2, KNTC1, KRT23, LAMA3, LEF1, LGR5, LINC00152, LINGO1, LPL, LRRC1, LYPD1, MAD2L1, MAGED4, MAGED4B, MAPK12, MAPK8IP2, MAPT, MCM2, MDGA1, MDK, MFAP2, MISP, MKI67, MMP11, MNS1, MPZ, MSC, MSH5, MTMR11, MUC13, MUC5B, MYBL2, MYH4, NAALADL1, NAV3, NCAPG, NDUFA4L2, NEB, NKD1, NMB, NOTCH3, NOTUM, NPM2, NQO1, NRCAM, NT5DC2, NTS, NUSAP1, OBSCN, OLFML2A, OLFML2B, OSBP2, PAQR4, PDZK1IP1, PEG10, PI3, PLCE1, PLCH2, PLK1, PLVAP, PLXDC1, PLXNB3, PODXL2, POLE2, PPAP2C, PRC1, PTGES, PTGFR, PTHLH, PTK7, PTP4A3, PTTG1, PYCR1, RACGAP1, RBM24, RECQL4, RHBG, RNF157, ROBO1, RP4-800G7.2, RPS6KL1, RRM2, S100A1, SCGN, 5-Sep, SERPINA12, SEZ6L2, SFN, SGOL2, SLC22A11, SLC51B, SLC6A2, SLC6A8, SLC6A9, SNCG, SOAT2, SP5, SPARCL1, SPINK1, SPP1, STIL, STK39, SULT1C2, TCF19, TDGF1, TESC, THY1, TK1, TMC5, TMEM132A, TMEM150B, TNFRSF19, TNFRSF25, TONSL, TOP2A, TPX2, TRIM16, TRIM16L, TRIM31, TRIM45, TTC39A, UBD, UBE2C, UBE2T, UGT2B11, USH1C, VSIG10L, WDR62, WDR76, ZWINT

The final data set used in all analyses consisted of log₂ (1+RPM) for the 248 transcripts and 83 samples identified as described above. Ten iterations of 10-fold cross-validation were implemented in order to evaluate the performance of the classification algorithm, which is described step-by-step below:
1. Feature selection. A one-sided t-test with alternative hypothesis H_{A}: μ_{CLD}<μ_{HCC} was conducted on the training set for each of the 248 transcripts identified by the TCGA differential expression analysis using the R stats package (version 3.4.2). Only those with p-values less than 0.05 were retained.
2. Random forest classifier. All transcripts kept from the feature selection step were used to train the random forest, which was built using the randomForest package (version 4.6-12) in R. The parameter m_{try} was left at its default value of sqrt(p), where p is the number of features in the data set, and nₜᵣₑₑ=500 trees were constructed. Sampling was stratified according to disease status. As a comparator classifier, a multivariable logistic regression model was created using the 10 most significant genes by p-value from the feature selection step.
3. Prediction. The proportion of trees in the random forest that voted for a classification of cancer for each sample in the test set were obtained from the random forest output and used to construct ROC curves with the pROC package (version 1.10.0).

### Example 2: Detection of CECs by Immunofluorescence

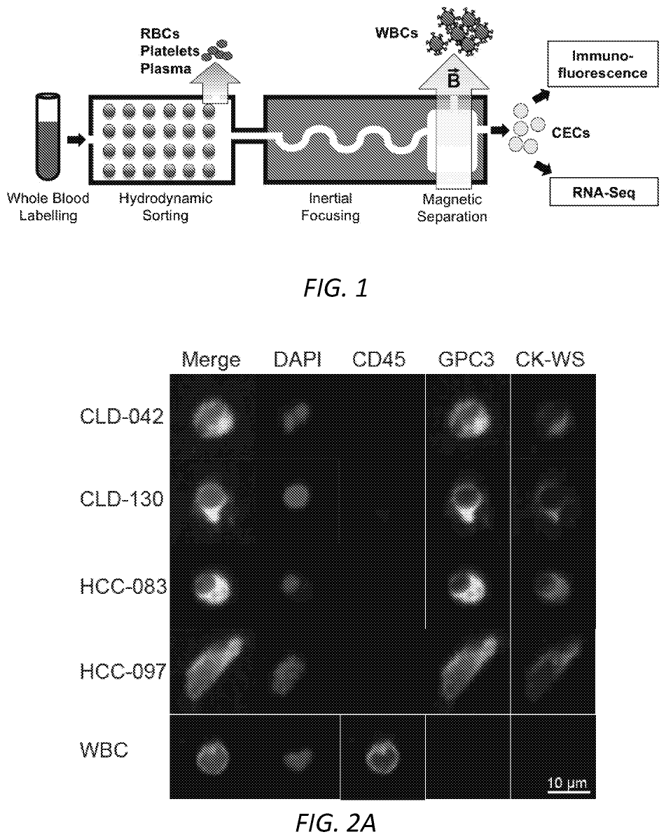

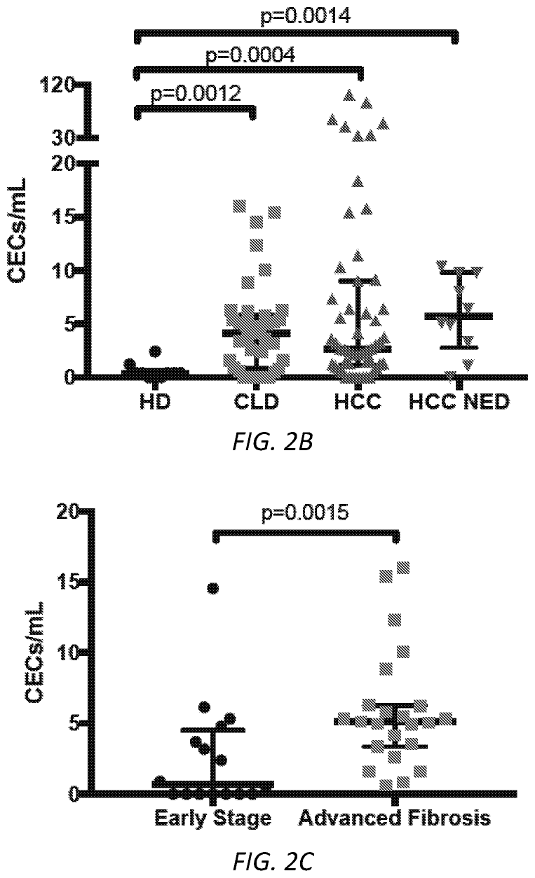

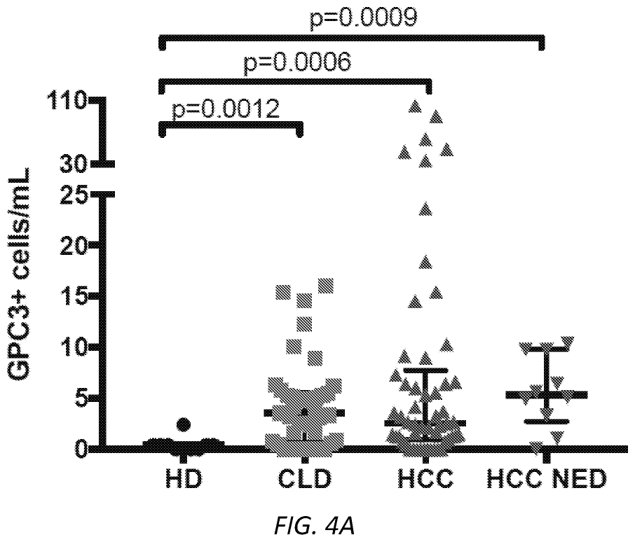

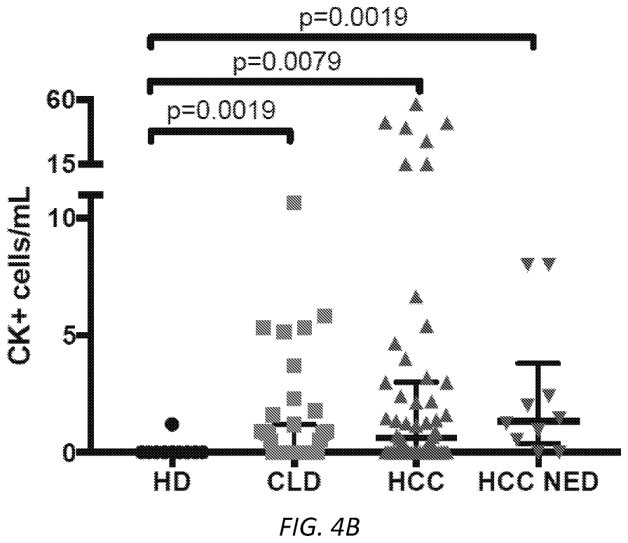

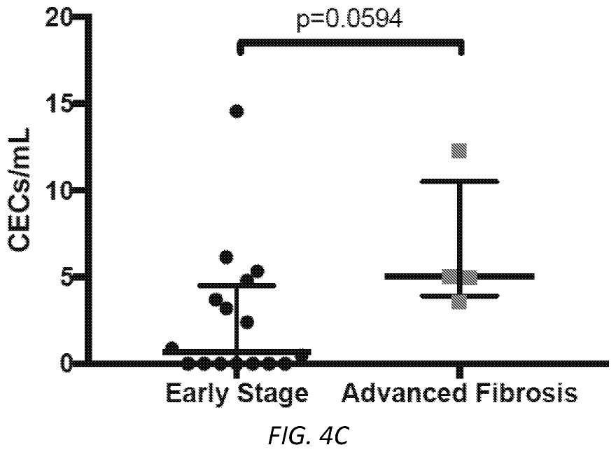

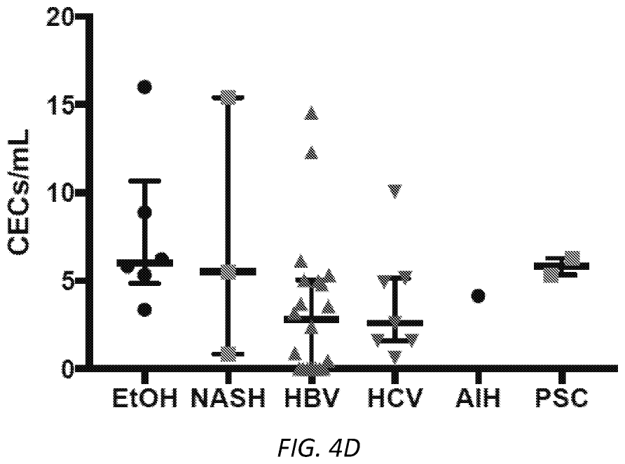

CECs were first detected by immunofluorescence (IF). Blood samples were obtained from 10 healthy blood donors, 39 CLD patients undergoing routine clinical surveillance for but had no evidence of HCC, 54 patients with HCC, and 10 HCC patients who underwent curative treatment and had no clinical evidence of disease (NED) (See Tables 1-4). The iChip device performed size-based exclusion of red blood cells, platelets and plasma, followed by magnetophoresis of labelled white blood cells (WBCs) (as described in Ozkumur E, et al. Sci Transl Med 2013; 5:179ra47) (see FIG. 1). CECs were then enumerated by IF staining for glypican-3, an oncofetal protein expressed in HCC but also in CLD liver tissue (as described in Wang H L, et al. Arch Pathol Lab Med 2008; 132:1723-8), or cytokeratin, an epithelial marker (see FIG. 2A). Using a threshold of 5 cells per 10 mL of whole blood, CECs were identified in a similar proportion of CLD patients (79%), HCC patients (810%), and NED patients (90%), but only in 20% of healthy donors (see FIG. 2B and FIGS. 4A-B; p<0.01, each group vs. healthy donors). Purification using the iChip device combined with immunofluorescent quantification demonstrated a high sensitivity for CEC detection with similar concentrations in HCC and CLD patients. Amongst CLD patients, those with advanced fibrosis (METAVIR F3 or F4) had a higher concentration of CECs (median 5.1 cells/mL) in comparison to those without advanced fibrosis (0.7 cells/mL, p<0.01, see FIG. 2C). Because the CLD study population consisted only of patients with sufficiently high risk of HCC to undergo surveillance, the etiology of CLD for each patient in the subgroup without advanced fibrosis was hepatitis B infection. The difference in CEC concentration associated with fibrosis stage did not appear to be due to CLD etiology, as the trend persisted when the analysis was restricted to only those with hepatitis B-induced CLD (median 5.0 cells/mL with advanced fibrosis, 0.7 cells/mL without advanced fibrosis, p=0.06, see FIG. 4C). Otherwise, there was no difference in CEC concentration by CLD etiology (see FIG. 4D).

### Example 3: Detection of CEC by RNA-Sequencing

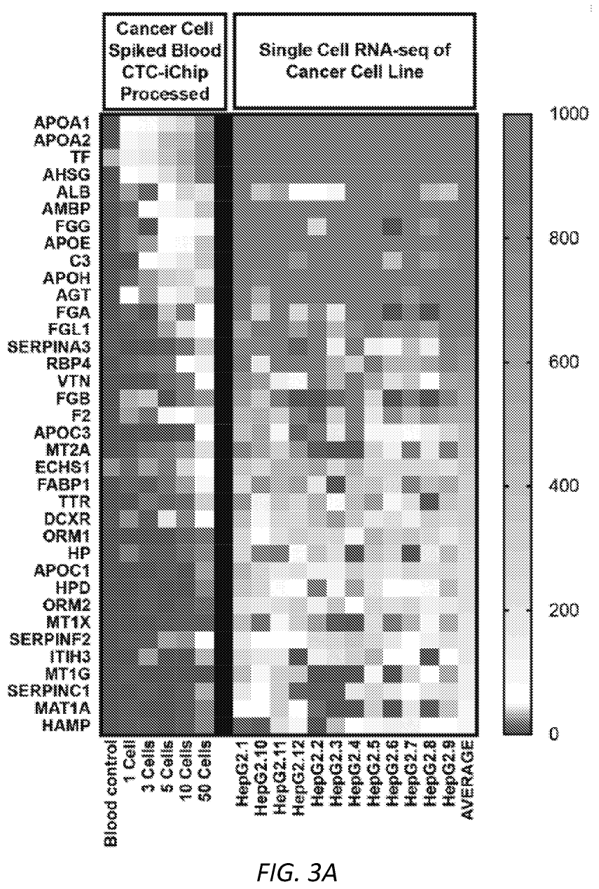

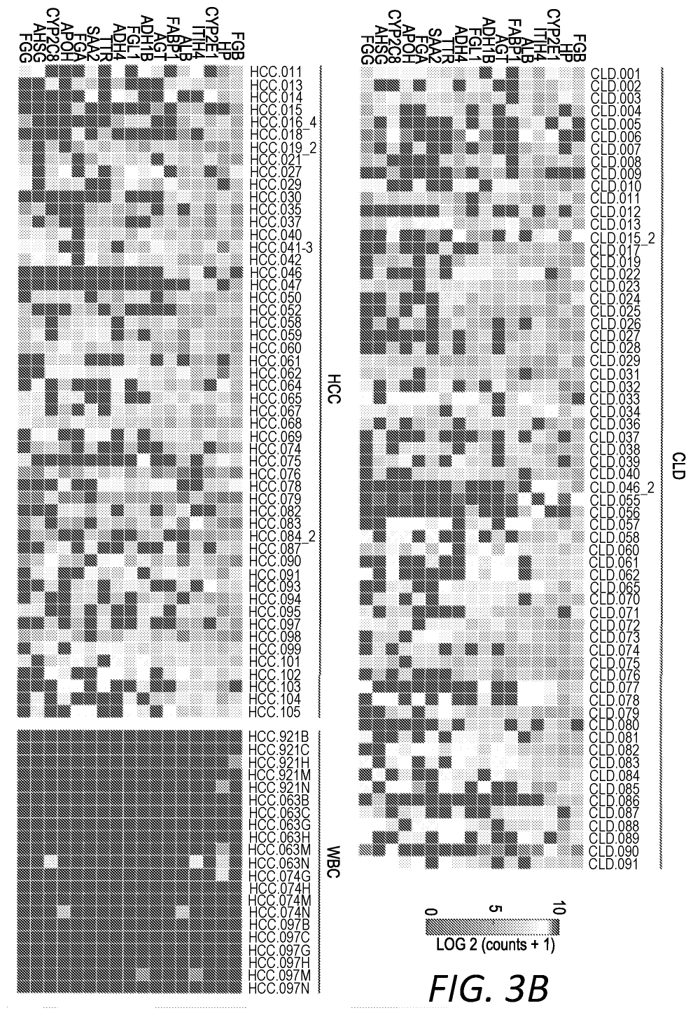

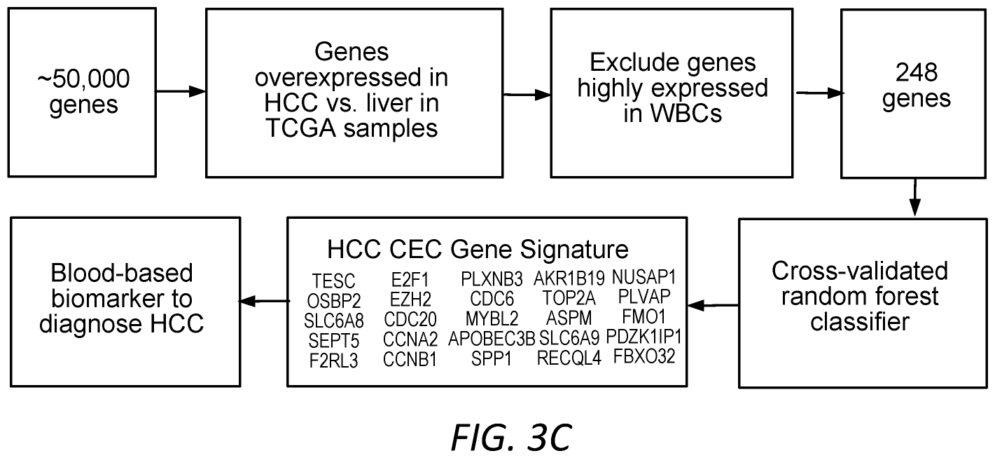

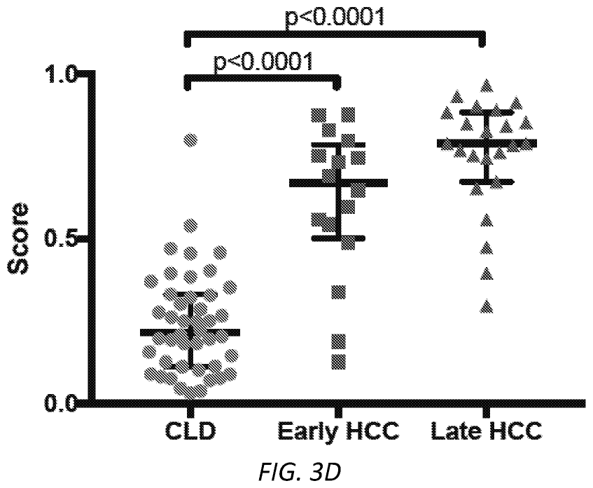

RNA-sequencing (RNA-seq) was performed to detect CECs. To determine the sensitivity of this approach, 0, 1, 3, 5, 10, or 50 HepG2 HCC cells were spiked into 4 mL of healthy donor blood and processed through the iChip device for RNA-seq. HepG2 specific gene expression was detectable in whole blood from a single cell (see FIG. 3A). CECs were identified in clinical blood samples from 64 CLD and 52 HCC patients. First, a 17 liver-specific gene signature was created based on Genotype Tissue Expression (GTEx) expression data. Liver-specific genes were identified in samples from both patient groups but were absent in WBC subtypes flow-sorted from the iChip device-processed blood (see FIG. 3B). Therefore, the liver-specific signature identified rare CECs rather than aberrant expression of these genes in contaminating WBCs.

### Example 4: Generation of Classifier for Detecting HCC

To show that CECs may phenotypically differ depending on the underlying disease state, gene expression profiling was performed to identify qualitative rather than quantitative differences between CECs in the setting of CLD versus HCC (see FIG. 3C). Using The Cancer Genome Atlas (TCGA) database, 248 genes were identified that were overexpressed in HCC compared to liver tissue and excluded genes expressed in WBCs. A Random Forest (RF) machine learning approach was used to generate a classifier based on these genes to distinguish CLD from HCC CECs. More specifically, each decision tree in the random forest casted a “vote” classifying a sample as CLD or HCC. The final classifier used 25 genes, which are listed in Table 5. Notably, three of the most informative genes in the classifier (TESC, SLC6A8, SPP1) have been implicated in cancer metastasis and another (E2F1) is an established cell proliferation marker (see Kang J, et al. Tumour Biol 2016; 37:13843-13853; Loo J M, et al. Cell 2015; 160:393-406; and Sangaletti S, et al. Cancer Res 2014; 74:4706-19).

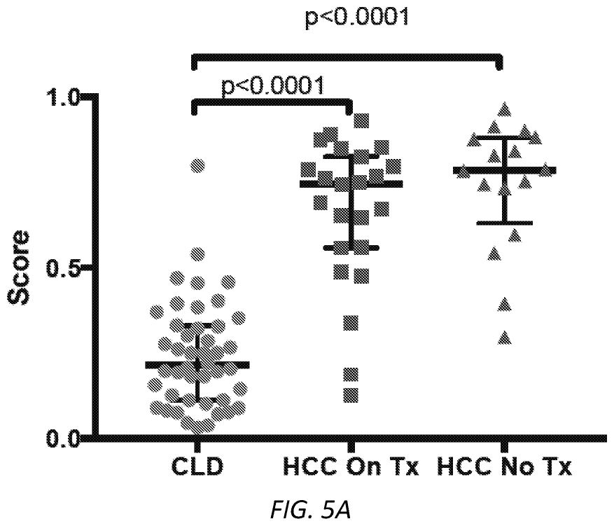

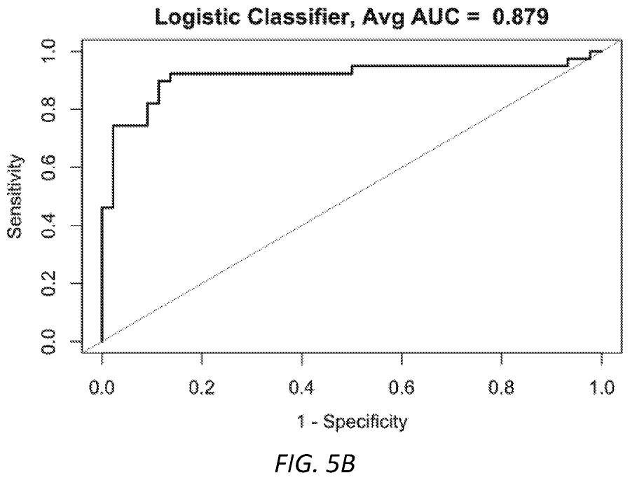

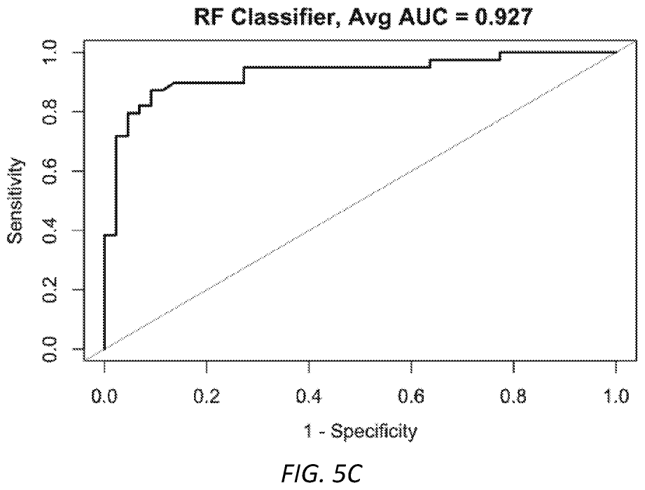

The cross-validated classifier provided excellent separation between CLD and HCC samples, with a sensitivity (i.e., true positive rate) of 85% at a specificity (i.e., true negative rate) of 95% and with identification of both early and late stage HCC (by Milan criteria) (see FIG. 3D and FIGS. 5A-C). The level of accuracy (sensitivity and specificity) achieved in this example is higher compared to a recent study (Cohen J D, et al. Science 2018) combining cell-free DNA and protein blood-based biomarkers, where an accuracy of only 44% for predicting HCC was achieved (may be due to the lack of common recurrent mutations and specific protein markers inherent to HCC).

TABLE 1
Demographics and results for CLD patients undergoing surveillance for HCC. CECs are defined
as cells expressing either CK or GPC3 by immunofluorescence. HCC Score is the vote fraction
from the RF classifier. HBV, hepatitis B virus; HCV, hepatitis C virus; PSC, primary sclerosing
cholangitis; NASH, non-alcoholic steatohepatitis; AIH (autoimmune hepatitis).

| | | | | | | CK+ | GPC3+ | CEC | |
|---|---|---|---|---|---|---|---|---|---|
| | | | | Advanced | AFP | (cells/ | (cells/ | (cells/ | HCC |
| Sample | Age | Sex | Diagnosis | Fibrosis | (ng/mL) | mL) | mL) | mL) | Score |
| CLD.001 | 61 | F | HCV | Yes | 2.7 | 0.0 | 10.1 | 10.1 | 0.39 |
| CLD.002 | 64 | M | HBV | No | — | 0.0 | 4.8 | 4.8 | 0.08 |
| CLD.003 | 31 | F | HBV | No | 3.3 | 0.0 | 14.5 | 14.5 | 0.21 |
| CLD.004 | 63 | M | Alcohol | Yes | 6.6 | 0.0 | 3.4 | 3.4 | 0.38 |
| CLD.005 | 81 | F | HBV | Yes | 1.9 | 0.0 | 5.0 | 5.0 | 0.40 |
| CLD.006 | 53 | M | HBV | Yes | 2.9 | 0.0 | 5.1 | 5.1 | 0.19 |
| CLD.007 | 36 | F | HBV | No | 2 | 0.9 | 5.3 | 5.3 | — |
| CLD.008 | 64 | M | HBV | No | 1.9 | 0.9 | 0.9 | 0.9 | 0.37 |
| CLD.009 | 59 | F | HBV | No | 3 | 0.8 | 3.2 | 3.2 | — |
| CLD.010 | 46 | F | Alcohol | Yes | 1.7 | 5.3 | 6.2 | 6.2 | 0.32 |
| CLD.011 | 77 | M | HBV | No | 1.7 | 0.0 | 0.0 | 0.0 | — |
| CLD.012 | 85 | F | Alcohol | Yes | 1.4 | 0.0 | 8.9 | 8.9 | — |
| CLD.013 | 87 | F | HCV | Yes | 8.1 | 0.9 | 2.6 | 2.6 | 0.14 |
| CLD.015_2 | 59 | M | HBV | No | 2.2 | — | — | — | 0.11 |
| CLD.017 | 66 | M | HCV | Yes | 4.4 | 0.0 | 1.6 | 1.6 | — |
| CLD.019 | 67 | M | Alcohol | Yes | 1.9 | 5.8 | 5.8 | 5.8 | — |
| CLD.020 | 40 | M | HBV | No | 2.5 | 0.5 | 0.5 | 0.5 | — |
| CLD.022 | 42 | M | PSC | Yes | 3.3 | 5.3 | 0.0 | 5.3 | 0.27 |
| CLD.023 | 72 | M | HCV | Yes | 1.7 | 0.8 | 1.6 | 1.6 | — |
| CLD.024 | 77 | M | HBV | Yes | 2.1 | 0.0 | 12.3 | 12.3 | 0.46 |
| CLD.025 | 54 | M | Alcohol/ | Yes | 10.4 | 10.7 | 15.4 | 15.4 | 0.54 |
| | | | NASH | | | | | | |
| CLD.026 | 55 | F | Alcohol | Yes | 4 | 0.0 | 5.3 | 5.3 | — |
| CLD.027 | 50 | M | HBV | No | 4.6 | 0.0 | 3.7 | 3.7 | — |
| CLD.028 | 70 | M | HBV | No | 3.2 | 0.0 | 0.0 | 0.0 | — |
| CLD.029 | 38 | M | PSC | Yes | 1.2 | 5.1 | 6.3 | 6.3 | — |
| CLD.030 | 59 | F | Crypto- | Yes | 5.5 | 0.9 | 16.0 | 16.0 | — |
| | | | genic | | | | | | |
| CLD.031 | 28 | M | HBV | No | 2.3 | 0.0 | 0.0 | 0.0 | — |
| CLD.032 | 70 | M | HCV | Yes | 3.2 | 0.0 | 0.6 | 0.6 | 0.33 |
| CLD.033 | 54 | M | HCV | Yes | 2.8 | — | — | — | 0.26 |
| CLD.034 | 73 | F | HBV | Yes | 2.8 | 1.8 | 3.6 | 3.6 | 0.46 |
| CLD.037 | 60 | F | HBV | No | 3.3 | 0.0 | 0.0 | 0.0 | — |
| CLD.038 | 60 | F | HBV | No | 2.1 | 3.7 | 3.1 | 6.2 | — |
| CLD.039 | 54 | F | HBV | No | 3.5 | 0.0 | 0.0 | 0.0 | 0.80 |
| CLD.040 | 39 | M | HBV | No | 4 | 0.0 | 0.0 | 0.0 | 0.22 |
| CLD.041 | 51 | M | HBV | No | 4.8 | 0.0 | 0.0 | 0.0 | — |
| CLD.042 | 54 | M | HBV | No | 3.2 | 1.6 | 2.4 | 2.4 | — |
| CLD.043 | 65 | F | NASH | Yes | 3.1 | 0.8 | 0.8 | 0.8 | — |
| CLD.044 | 57 | M | NASH | Yes | 4 | 0.6 | 5.5 | 5.5 | — |
| CLD.045 | 68 | M | HCV/ | Yes | 12.2 | 0.0 | 4.9 | 4.9 | — |
| | | | NASH | | | | | | |
| CLD.046_2 | 60 | M | HBV | Yes | 3.2 | — | — | — | 0.35 |
| CLD.048 | 66 | F | HCV | Yes | 3.1 | 2.3 | 5.1 | 5.1 | — |
| CLD.050 | 50 | F | AIH | Yes | 5 | 1.2 | 4.1 | 4.1 | — |
| CLD.055 | 37 | F | HBV | No | 1.4 | — | — | — | 0.30 |
| CLD.056 | 47 | M | HBV | No | 4.4 | — | — | — | 0.25 |
| CLD.057 | 44 | F | HBV | No | 1.6 | — | — | — | 0.09 |
| CLD.058 | 31 | M | HBV | No | 1.7 | — | — | — | 0.18 |
| CLD.060 | 61 | F | AIH | Yes | 10.5 | — | — | — | 0.08 |
| CLD.061 | 69 | F | HCV | Yes | 3.2 | — | — | — | 0.04 |
| CLD.062 | 42 | F | HBV | No | 4.8 | — | — | — | 0.05 |
| CLD.065 | 54 | M | HCV | Yes | 2.4 | — | — | — | 0.07 |
| CLD.070 | 45 | F | HBV | No | 4.8 | — | — | — | 0.25 |
| CLD.071 | 66 | M | HBV | No | 4.8 | — | — | — | 0.32 |
| CLD.072 | 48 | F | HBV | No | 1.3 | — | — | — | 0.18 |
| CLD.073 | 69 | F | HCV | Yes | 4.6 | — | — | — | 0.20 |
| CLD.077 | 44 | M | HBV | Yes | 5.1 | — | — | — | 0.10 |
| CLD.078 | 40 | M | HCV | No | 1.3 | — | — | — | 0.16 |
| CLD.079 | 54 | M | HBV | No | 1.6 | — | — | — | 0.28 |
| CLD.081 | 74 | M | HBV | No | 2 | — | — | — | 0.29 |
| CLD.082 | 74 | M | NASH | Yes | 1.9 | — | — | — | 0.47 |
| CLD.083 | 60 | F | HBV | No | 4.4 | — | — | — | 0.25 |
| CLD.084 | 69 | M | HBV | Yes | 3.2 | — | — | — | 0.11 |
| CLD.085 | 64 | M | HBV | Yes | 2.6 | — | — | — | 0.20 |
| CLD.087 | 64 | M | HBV | Yes | 2.6 | — | — | — | 0.08 |
| CLD.088 | 69 | M | HCV | Yes | 4.4 | — | — | — | 0.09 |
| CLD.089 | 43 | M | HBV | No | 6.6 | — | — | — | 0.13 |
| CLD.090 | 69 | M | HCV | Yes | 4.4 | — | — | — | 0.20 |
| CLD.091 | 43 | M | HBV | No | 6.6 | — | — | — | 0.03 |

TABLE 2
Demographics and results for HCC patients with active disease (with or without treatment prior to blood
draw). CECs are defined as cells expressing either CK or GPC3 by immunofluorescence. HCC Score is the
vote fraction from the RF classifier. NASH, non-alcoholic steatohepatitis; PSC, primary sclerosing cholangitis;
HBV, hepatitis B virus; HCV, hepatitis C virus; A1AT alpha-1-antitrypsin deficiency; RT, radiotherapy
(external); TACE, transarterial chemoembolization; SIRT, selective internal radiation therapy.

| | | | | | | | | CK+ | GPC3+ | CECs | |
|---|---|---|---|---|---|---|---|---|---|---|---|
| | | | Risk | | AFP | | Milan | (cells/ | (cells/ | (cells/ | HCC |
| Sample | Age | Sex | Factor | Cirrhosis | (ng/mL) | Treatment | Criteria | mL) | mL) | mL) | Score |
| HCC.008 | 43 | M | Hemo- | No | 9534 | RT, | No | 57 | 103.5 | 103.5 | — |
| | | | chromatosis | | | sorafenib, | | | | | |
| | | | | | | chemo, | | | | | |
| | | | | | | checkpoint | | | | | |
| | | | | | | inhibitor, | | | | | |
| | | | | | | resection | | | | | |
| HCC.011 | 74 | M | NA | No | 939.2 | Ablation, | No | 2.4 | 18.4 | 18.4 | 0.32 |
| | | | | | | resection | | | | | |
| HCC.013_0 | 63 | F | Budd | Yes | 6.1 | None | Yes | 31.1 | 90.4 | 90.4 | — |
| | | | Chiari | | | | | | | | |
| HCC.013 | 63 | F | Budd | Yes | 3.4 | Ablation, RT | Yes | — | — | — | 0.87 |
| | | | Chiari | | | | | | | | |
| HCC.014 | 69 | F | NASH | Yes | 218 | TACE | Yes | 0.7 | 0.7 | 0.7 | 0.24 |
| HCC.015 | 70 | F | PSC | Yes | 3.8 | None | Yes | 3 | 0 | 3 | 0.78 |
| HCC.016 | 70 | M | HBV | No | 101.5 | Thymalfasin | Yes | 0 | 0 | 0 | — |
| HCC.018 | 82 | M | NASH | No | 132367 | None | Yes | 40.6 | 61.8 | 61.8 | 0.86 |
| HCC.019 | 64 | M | Alcohol | Yes | 10.4 | None | No | 44 | 45.1 | 55.4 | — |
| HCC.019_2 | 64 | M | Alcohol | Yes | 21 | RT, sorafenib | No | — | — | — | 0.85 |
| HCC.021 | 68 | M | NASH, | Yes | 4891 | Ablation | No | 0 | 4.2 | 4.2 | — |
| | | | alcohol | | | | | | | | |
| HCC.025 | 68 | M | Alcohol | Yes | 4.3 | None | Yes | 0.9 | 1.7 | 2.6 | — |
| HCC.026 | 85 | M | Crypto- | Yes | 1.3 | None | Yes | 0 | 15.4 | 15.4 | — |
| | | | genic | | | | | | | | |
| HCC.027 | 55 | M | HBV | No | 731.8 | None | No | 5.4 | 6.6 | 11.4 | 0.83 |
| HCC.029 | 70 | M | NASH | Yes | 4800 | Ablation | No | 15.2 | 23.6 | 34.2 | 0.80 |
| HCC.030_0 | 78 | M | HBV | Yes | 151.6 | None | No | 3 | 9 | 9 | — |
| HCC.030 | 78 | M | HBV | Yes | 26.9 | TACE | Yes | — | — | — | 0.42 |
| HCC.031_0 | 63 | M | HCV | Yes | 64.9 | Ablation, | NED | 15 | 34.5 | 35.3 | — |
| | | | | | | TACE, | | | | | |
| | | | | | | transplant | | | | | |
| HCC.034 | 71 | F | NA | No | 3.8 | None | No | 0.6 | 3.6 | 3.6 | — |
| HCC.035 | 82 | F | NA | No | 2043.5 | SIRT, RT, | No | 1.1 | 9.1 | 9.1 | — |
| | | | | | | sorafenib | | | | | |
| HCC.037 | 54 | M | Alcohol | Yes | 5947 | Ablation, | No | 0 | 3.3 | 3.3 | — |
| | | | | | | SIRT, | | | | | |
| | | | | | | sorafenib | | | | | |
| HCC.040_0 | 70 | M | NA | No | 2.2 | Resection | No | 1.3 | 14.5 | 15.8 | — |
| HCC.040 | 70 | M | NA | No | 5.6 | RT, | No | NA | NA | NA | 0.77 |
| | | | | | | checkpoint | | | | | |
| | | | | | | inhibitor, | | | | | |
| | | | | | | resection | | | | | |
| HCC.041 | 72 | M | Alcohol | Yes | 338 | RT | No | 0 | 1.2 | 1.2 | — |
| HCC.041_3 | 72 | M | Alcohol | Yes | 1092 | RT, sorafenib | No | — | — | — | 0.52 |
| HCC.042 | 58 | M | HCV, | Yes | 5.4 | None | No | 0.5 | 0.5 | 0.5 | 0.43 |
| | | | alcohol | | | | | | | | |
| HCC.044 | 79 | F | NA | No | 19598 | None | No | 0.5 | 0 | 0.5 | — |
| HCC.046 | 66 | M | HBV | Yes | 5.5 | Ablation | Yes | 1.4 | 10.3 | 10.3 | 0.05 |
| HCC.047 | 62 | M | Alcohol | Yes | 12.7 | Ablation | Yes | 0 | 0 | 0 | — |
| HCC.050 | 76 | M | Alcohol | Yes | 5.4 | Ablation, | Yes | 0 | 2.3 | 2.3 | — |
| | | | | | | TACE, RT | | | | | |
| HCC.052 | 23 | M | Biliary | Yes | 1.5 | RT | No | 0 | 2.5 | 2.5 | — |
| | | | atresia | | | | | | | | |
| HCC.059 | 66 | M | HCV, | Yes | 4847 | Ablation | No | 43.6 | 48.7 | 49.5 | 0.91 |
| | | | alcohol | | | | | | | | |
| HCC.060 | 63 | F | NASH | Yes | 13629 | None | No | 0 | 3.2 | 3.2 | 0.77 |
| HCC.061 | 67 | F | NA | No | 60.6 | Reection | No | 0 | 1.3 | 1.3 | — |
| HCC.062 | 63 | M | NASH | Yes | 185.2 | TACE | Yes | 0 | 3.7 | 3.7 | 0.71 |
| HCC.064 | 53 | M | HCV | Yes | 1.6 | None | Yes | 0 | 0.8 | 0.8 | 0.85 |
| HCC.065 | 59 | M | HBV | No | 63.3 | None | Yes | 0 | 0 | 0 | 0.80 |
| HCC.067 | 74 | F | NA | No | 2.5 | Sorafenib | No | 0.7 | 1.3 | 1.3 | 0.89 |
| HCC.068 | 83 | M | HBV | Yes | 4.7 | RT | No | NA | NA | NA | 0.79 |
| HCC.069 | 62 | M | NASH | Yes | 20.8 | TACE | Yes | NA | NA | NA | 0.88 |
| HCC.074_0 | 64 | M | NASH, | Yes | 167580 | None | No | 0 | 0 | 0 | — |
| | | | alcohol | | | | | | | | |
| HCC.075 | 81 | M | HBV | Yes | 7.7 | Ablation, | No | 0 | 1 | 1 | 0.76 |
| | | | | | | soracenib, | | | | | |
| | | | | | | chemo, | | | | | |
| | | | | | | checkpoint | | | | | |
| | | | | | | inhibitor, | | | | | |
| | | | | | | resection | | | | | |
| HCC.076 | 79 | M | HBV | Yes | 13322 | None | No | 0 | 1.5 | 1.5 | — |
| HCC.078 | 60 | M | HBV | No | 4.7 | Ablation, | No | 2.2 | 2.2 | 2.2 | 0.83 |
| | | | | | | resection | | | | | |
| HCC.079 | 71 | M | NA | No | 5.2 | None | No | — | — | — | 0.73 |
| HCC.082 | 62 | M | HCV, | Yes | 5.9 | None | Yes | 6.7 | 7.3 | 7.3 | 0.80 |
| | | | alcohol | | | | | | | | |
| HCC.083 | 59 | M | HCV, | Yes | 8.5 | TACE, SIRT, | No | 2.1 | 2.1 | 2.1 | 0.89 |
| | | | alcohol | | | RT, sorafenib | | | | | |
| HCC.084 | 81 | M | NA | No | 16.8 | Sorafenib | No | 0 | 0 | 0 | — |
| HCC.087 | 57 | M | Alcohol | Yes | 16.7 | Ablation, | Yes | 0 | 1.2 | 1.2 | 0.80 |
| | | | | | | TACE | | | | | |
| HCC.090 | 69 | M | Alcohol | Yes | 19960 | None | No | 4.7 | 5.3 | 5.3 | 0.92 |
| HCC.091 | 72 | M | HBV | No | 3.2 | None | Yes | 0 | 0 | 0 | 0.91 |
| HCC.093 | 77 | M | NA | No | 3.2 | RT, resection | No | 0 | 0 | 0 | 0.80 |
| HCC.094 | 70 | M | NA | No | 156.4 | SIRT, RT | No | 4 | 6 | 6 | — |
| HCC.095 | 64 | M | HCV, | Yes | 7.6 | TACE | Yes | 1.3 | 2.7 | 2.7 | 0.66 |
| | | | alcohol | | | | | | | | |
| HCC.097 | 52 | F | NA | No | 1.3 | Ablation, | No | 0 | 6.4 | 6.4 | 0.66 |
| | | | | | | chemo, | | | | | |
| | | | | | | transplant, | | | | | |
| | | | | | | resection, | | | | | |
| | | | | | | everolimus/ | | | | | |
| | | | | | | leuprolide, | | | | | |
| | | | | | | sitravatinib | | | | | |
| HCC.098 | 66 | M | Alcohol | Yes | 356 | None | No | 0 | 0 | 0 | 0.65 |
| HCC.099 | 58 | M | Alcohol | Yes | 254.9 | TACE | Yes | 1.5 | 1.5 | 1.5 | 0.51 |
| HCC.101 | 78 | M | NASH | Yes | NA | None | Yes | 1.2 | 2.5 | 2.5 | 0.54 |
| HCC.102 | 61 | M | Alcohol, | Yes | 9.6 | None | Yes | 0 | 5.6 | 5.6 | 0.87 |
| | | | Hemo- | | | | | | | | |
| | | | chromatosis | | | | | | | | |
| HCC.103 | 68 | M | A1AT | Yes | 286.8 | Ablation | Yes | 0 | 2.7 | 2.7 | 0.59 |
| HCC.104 | 74 | M | NA | No | 15.4 | None | No | 3.2 | 6.4 | 6.4 | 0.49 |
| HCC.105 | 56 | M | HCV | Yes | 3.7 | None | No | 1.6 | 1.6 | 1.6 | 0.89 |

TABLE 3
Demographics and results for HCC patients with no evidence of disease after treatment.
CECs are defined as cells expressing either CK or GPC3 by immunofluorescence. NASH,
non-alcoholic steatohepatitis; HBV, hepatitis B virus; HCV, hepatitis C virus.

| | | | | | | | CK+ | GPC3+ | CECs |
|---|---|---|---|---|---|---|---|---|---|
| | | | Risk | | AFP | | (cells/ | (cells/ | (cells/ |
| Sample | Age | Sex | Factor | Cirrhosis | (ng/mL) | Treatment | mL) | mL) | mL) |
| HCC.033_0 | 85 | M | Hemo- | No | 1.3 | TACE | 2.0 | 3.3 | 3.3 |
| | | | chromatosis | | | | | | |
| HCC.051 | 68 | M | NASH | Yes | 2.5 | Ablation, | 0.9 | 10.4 | 10.4 |
| | | | | | | TACE, | | | |
| | | | | | | transplant | | | |
| HCC.053 | 63 | M | HCV, | Yes | 2.3 | Resection | 0.0 | 6.4 | 6.4 |
| | | | alcohol | | | | | | |
| HCC.055 | 51 | M | Alcohol | Yes | 2.6 | Ablation, | 2.4 | 5.6 | 8.0 |
| | | | | | | transplant | | | |
| HCC.058_2 | 54 | M | HBV | Yes | 3.6 | Resection | 0.0 | 0.0 | 0.0 |
| HCC.063 | 68 | M | HBV | Yes | 22.3 | TACE | 8.0 | 9.8 | 9.8 |
| HCC.077 | 77 | M | Budd | No | 16.7 | Ablation | 8.0 | 9.8 | 9.8 |
| | | | Chiari | | | | | | |
| HCC.085 | 71 | M | NASH | Yes | 7.4 | TACE | 1.2 | 4.9 | 4.9 |
| HCC.086 | 75 | M | HBV | Yes | 2.9 | Ablation | 0.5 | 1.1 | 1.1 |
| HCC.088 | 81 | M | HCV | No | 1.6 | Ablation | 1.5 | 5.1 | 5.1 |

TABLE 4
Healthy blood donor demographics.

| | | | CK+ | GPC3+ | CECs |
|---|---|---|---|---|---|
| Sample | Gender | Age | (cells/mL) | (cells/mL) | (cells/mL) |
| HD.01 | F | 28 | 0 | 0.4 | 0.4 |
| HD.02 | M | 34 | 0 | 0.4 | 0.4 |
| HD.03 | M | 40 | 0 | 0.4 | 0.4 |
| HD.04 | M | 29 | 0 | 2.4 | 2.4 |
| HD.05 | M | 23 | 0 | 0 | 0 |
| HD.06 | M | 38 | 0 | 0 | 0 |
| HD.07 | F | 24 | 0 | 0.4 | 0.4 |
| HD.08 | F | 26 | 0 | 0.4 | 0.4 |
| HD.09 | F | 35 | 1.2 | 0 | 1.2 |
| HD.10 | F | 27 | 0 | 0 | 0 |

TABLE 5
Gene signature for blood-based biomarker to diagnose HCC. Gene weight is the mean
decrease in Gini, as a metric for the contribution of the gene to the classifier.

| | | | Involvement in | |
|---|---|---|---|---|
| Gene | Weight | Gene Function | Cancer | Publication |
| TESC | 5.170 | Functions as an integral | Metastasis in | Kang J, et al. |
| | | cofactor in cell pH | Colorectal Cancer | Tumour Biol 2016 |
| | | regulation by | | |
| | | controlling plasma | | |
| | | membrane-type | | |
| | | Na+/H+ exchange | | |
| | | activity. | | |
| OSBP2 | 4.203 | Lipid binding protein | Carcinogenesis via | Du X, et al. Semin |
| | | | ERK pathway | Cell Dev Biol |
| | | | | 2018 |
| SLC6A8 | 3.937 | Required for the uptake | Increases survival | Loo J M, et al. Cell |
| | | of creatine in muscles | of metastases | 2015 |
| | | and brain. | | |
| SEPT5 | 2.504 | Cytokinesis and vesicle | Carcinogenesis | Russell S, et al. Br |
| | | trafficking | | J Cancer 2005 |
| F2RL3 | 1.502 | Protease-activated | Methylation status | Zhang Y, et al. Int |
| | | receptor involved in | associated with | J Cancer 2015 |
| | | transmembrane | lung cancer risk and | |
| | | signaling | morality | |
| E2F1 | 1.378 | Cell cycle control | Proliferation | Zhan L, et al. Cell |
| | | | | Signal 2014 |
| EZH2 | 1.079 | Regulates | Altered | Kim K, et al. Nat |
| | | transcriptional | transcriptional | Med 2016 |
| | | repression | programming | |
| CDC20 | 0.924 | Cell cycle control | Proliferation | Kidokoro T, et al. |
| | | | | Oncogene 2008 |
| CCNA2 | 0.894 | Cell cycle control | Proliferation | Gao T, et al. PLOS |
| | | | | One 2014 |
| CCNB1 | 0.876 | Cell cycle control | Proliferation, | Patil M, et al. |
| | | | hepatocarcinognesis | Cancer Res 2009 |
| PLXNB3 | 0.766 | Cell migration | Unclear | |
| CDC6 | 0.754 | Cell cycle control | Carcinogenesis | Yao Z, et al. |
| | | | | Cancer Biol Ther |
| | | | | 2009 |
| MYBL2 | 0.689 | Cell cycle control | Proliferation, | Musa J, et al. Cell |
| | | | survival | Death Dis 2017 |
| APOBEC3B | 0.653 | Cytadine deaminase | Mutagenesis | Kuong K, et al. |
| | | | | Nat Genet 2014 |
| SPP1 | 0.648 | ECM protein important | Involved with | Sangaletti S, et al. |
| | | for tissue remodeling. | metastasis | Cancer Research |
| | | Also acts as cytokine. | | 2014 |
| AKR1B10 | 0.639 | Aldo-keto reductase | Mediates liver | Jin J, et al. |
| | | | cancer cell | Scientific Reports |
| | | | proliferation | 2016 |
| TOP2A | 0.606 | Topoisomerase | Carcinogenesis | Wong N, et al. Int |
| | | | | J Cancer 2009 |
| ASPM | 0.600 | Mitotic spindle | Marker of | Lin S Y, et al. Clin |
| | | regulation | invasiveness in | Cancer Res 2008 |
| | | | HCC | |
| SLC6A9 | 0.579 | Sodium-dependent | Unclear | |
| | | reuptake of glycine | | |
| RECQL4 | 0.554 | Human DNA helicases | Upregulation poor | Li J, et al. |
| | | involved in genomic | prognosis in HCC | Oncology Letters |
| | | instability | | 2017 |
| NUSAP1 | 0.554 | Spindle microtubule | Involved with | Gordon C, et al. |
| | | organization | invasion and | Oncotarget 2017 |
| | | | metastasis | |
| PLVAP | 0.540 | Involved in the | Induced in | Carson-Walter E B, |
| | | formation of stomatai | endothelium of | et al. Clin Cancer |
| | | and fenestral | cancers with | Research 2005 |
| | | diaphragms of caveolae. | enhanced | |
| | | | metastasis and | |
| | | | angiogenesis | |
| FMO1 | 0.523 | Oxidative metabolism | Unclear | |
| | | of xenobiotics | | |
| PDZK1IP1 | 0.520 | Intracellular protein | Regulation of | Garcia-Heredia |
| | | trafficking | immune | J M, et al. |
| | | | microenvironment | Oncotarget 2017 |
| FBXO32 | 0.510 | Substrate recognition | Unclear | |
| | | for ubiquitination | | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method, comprising measuring expression levels of hepatocellular carcinoma (HCC) classifier genes in circulating epithelial cells (CECs) of a subject, wherein the HCC classifier genes comprise one or more of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

2. The method of claim 1, wherein the HCC classifier genes consist of one or more of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

3. The method of claim 1 or 2, wherein the HCC classifier genes consist of TESC, OSBP2, SLC6A8, SEPT5, F2RL3, E2F1, EZH2, CDC20, CCNA2, CCNB1, PLXNB3, CDC6, MYBL2, APOBEC3B, SPP1, AKR1B10, TOP2A, ASPM, SLC6A9, RECQL4, NUSAP1, PLVAP, FMO1, PDZK1IP1, and FBXO32.

4. The method of claim 1, wherein the HCC classifier genes further comprise one, two, three or more additional genes selected from the group consisting of ACTG2, ADM2, AFP, AGR2, ALDH3A1, ALPK3, AMIGO3, ANKRD65, ANLN, AP1M2, ARHGAP11A, ARHGEF39, ASF1B, ASPHD1, AURKA, AXIN2, BAIAP2L2, BEX2, C15orf48, C1orf106, C1QTNF3, C6orf223, CA12, CA9, CAMK2N2, CAP2, CBX2, CCDC170, CCDC28B, CCDC64, CCNE2, CCNF, CD109, CD34, CDC25A, CDC7, CDCA5, CDCA8, CDH13, CDK1, CDKN2A, CDKN2C, CDT1, CELF6, CENPF, CENPH, CENPL, CENPU, CENPW, CKB, CNNM1, COL15A1, COL4A5, COL7A1, COL9A2, CRIP3, CSPG4, CTNND2, CXorf36, CYP17A1, DLK1, DMKN, DSCC1, DTL, DUOX2, ECT2, EEF1A2, EFNA3, EPHB2, EPPK1, ETV4, FABP4, FAM111B, FAM3B, FAM83D, FANCD2, FANCI, FBXL18, FERMT1, FGF19, FLNC, FLVCR1, FOXD2-AS1, FOXM1, FXYD2, GABRE, GAL3ST1, GCNT3, GINS1, GJC1, GMNN, GNAZ, GOLGA2P7, GPC3, GPR64, GPSM1, HRCT1, IGF2BP2, IGSF1, IGSF3, IQGAP3, ITGA2, ITPKA, KIAA0101, KIF11, KIFC1, KIFC2, KNTC1, KRT23, LAMA3, LEF1, LGR5, LINC00152, LINGO1, LPL, LRRC1, LYPD1, MAD2L1, MAGED4, MAGED4B, MAPK12, MAPK8IP2, MAPT, MCM2, MDGA1, MDK, MFAP2, MISP, MKI67, MMP11, MNS1, MPZ, MSC, MSH5, MTMR11, MUC13, MUC5B, MYH4, NAALADL1, NAV3, NCAPG, NDUFA4L2, NEB, NKD1, NMB, NOTCH3, NOTUM, NPM2, NQO1, NRCAM, NT5DC2, NTS, OBSCN, OLFML2A, OLFML2B, PAQR4, PEG10, PI3, PLCE1, PLCH2, PLK1, PLXDC1, PODXL2, POLE2, PPAP2C, PRC1, PTGES, PTGFR, PTHLH, PTK7, PTP4A3, PTTG1, PYCR1, RACGAP1, RBM24, RHBG, RNF157, ROBO1, RP4-800G7.2, RPS6KL1, RRM2, S100A1, SCGN, 5-Sep, SERPINA12, SEZ6L2, SFN, SGOL2, SLC22A11, SLC51B, SLC6A2, SNCG, SOAT2, SP5, SPARCL1, SPINK1, STIL, STK39, SULT1C2, TCF19, TDGF1, THY1, TK1, TMC5, TMEM132A, TMEM150B, TNFRSF19, TNFRSF25, TONSL, TPX2, TRIM16, TRIM16L, TRIM31, TRIM45, TTC39A, UBD, UBE2C, UBE2T, UGT2B11, USH1C, VSIG10L, WDR62, WDR76, and ZWINT.

5. A method for detecting the presence of HCC in a subject having chronic liver disease (CLD), the method comprising:
(a) measuring expression levels of the HCC classifier genes of any one of claims 1-4 in CECs of the subject; and
(b) comparing the expression levels of the HCC classifier genes in the CECs of the subject with reference expression levels of HCC classifier genes thereby determining the presence of HCC.

6. The method of claim 5, wherein the expression levels of HCC classifier genes are used to calculate a HCC score, and the calculated HCC score is compared with a reference score, wherein the presence of HCC is determined based on the presence of a HCC score above the reference score.

7. The method of claim 6, wherein the HCC score is calculated using a random forest analysis.

8. The method of claim 5, wherein the expression levels of HCC classifier genes are compared with the reference expression levels of HCC classifier genes using a multivariate logistic regression modeling approach.

9. The method of any one of claims 1-8, wherein the expression levels of HCC classifier genes in circulating epithelial cells (CECs) are measured by:
(a) obtaining a sample comprising blood from the subject;
(b) removing red blood cells, platelets, and plasma from the sample by size-based exclusion;
(c) removing white blood cells (WBCs) from the sample by magnetophoresis; and
(d) measuring the expression of a set of genes in the CECs using RNA-sequencing, qRT-PCT, RNA in situ hybridization, protein microarray, or mass spectrometry and protein profiling.

10. The method of any one of claims 5-9, wherein the HCC being detected is an early stage HCC.

11. The method of any one of claims 5-9, wherein the HCC being detected is a late stage HCC.

12. The method of any one of claims 5-11 further comprising:
(a) confirming or having confirmed the presence of HCC in the patient by ultrasound imaging, dynamic CT, MRI imaging, needle biopsy, and/or biopsy; and
(b) if the presence of HCC in the patient is confirmed, treating or having the subject treated for HCC by surgical removal of the HCC tissue, radiofrequency ablation of the HCC tissue, embolization of the HCC tissue; embolization of HCC tissue, chemotherapy, and/or cryotherapy.

13. A method of monitoring a subject having CLD for development of HCC, the method comprising:
(a) performing the method of claim 6 or 7 at an initial time point, and if the HCC score is below the reference score, then
(b) performing the method of claim 6 or 7 at one or more subsequent time points.

14. The method of claim 13, wherein step (b) is performed at one or more subsequent time points until the presence of HCC is determined.

15. The method of claim 13 or 14, wherein the initial and each subsequent time point is about three months, six months, or a year apart.

16. A method of distinguishing between the presence of early stage liver fibrosis and late stage liver fibrosis in a subject having CLD, the method comprising:
(a) detecting a concentration of CECs in a blood sample of the subject;
(b) comparing the concentration of CECs in the blood sample of the subject with a reference value;
(c) diagnosing the subject with early stage fibrosis if the subject has concentration of CECs in the blood sample that is below the reference value; and
(d) diagnosing the subject with late stage fibrosis if the subject has concentration of CECs in the blood sample that is above the reference value.

17. The method of claim 16, wherein the subject has hepatitis B.

18. The method of claim 16 or 17, wherein the concentration of CECs is measured by immunofluorescence.

19. The method of any one of claims 16-18, wherein the concentration of CECs is measured by detecting glypican-3 (GPC3) and/or cytokeratins (CKs).

20. A method of monitoring a subject having CLD for development of advanced fibrosis, the method comprising:
(a) performing the method of any one of claims 16-19; and if the concentration of CECs in the blood sample of the subject is lower than the reference value, then
(b) performing the method of any one of claims 16-19 at one or more subsequent time points.

21. The method of claim 20, wherein step (b) is performed at one or more subsequent time points until the subject is diagnosed with late stage fibrosis.

22. The method of any one of claims 16-20, wherein the initial and each subsequent time point is about three months, six months, or a year apart.

23. A method of monitoring a subject having CLD being treated to prevent the progression of fibrosis or HCC, the method comprising:
(a) performing the method of any one of claims 16-19; and if the concentration of CECs in the blood sample of the subject is lower than the reference value, then performing the method of any one of claims 16-19 at one or more subsequent time point; and
(b) performing the method of claim 6 or 7 at an initial time point, and if the expression levels of the HCC score is below the reference score, then performing the method of claim 6 or 7 at one or more subsequent time points.

24. The method of claim 23, wherein step (a) is performed at one or more subsequent time points until the subject is diagnosed with late stage fibrosis, and/or wherein step (b) is performed at one or more subsequent time points until the presence of HCC is determined.

25. The method of claim 24, wherein the first initial and each subsequent time point for performing step (a) or step (b) of claim 23 is about three months, six months, or a year apart, and the second initial and each subsequent time point is about three months, six months, or a year apart.

26. The method of any one of claims 1-25, wherein the CECs in the blood are purified or enriched using a microfluidic device.

27. The method of claim 26, wherein the microfluidic device is an iChip device.
